(19) United States
(12) Patent Application Publication
(10) Pub. No.: US 2018/0258411 A1
(43) Pub. Date: Sep. 13, 2018

(54) COMPOSITIONS AND METHODS FOR GENOME EDITING

(71) Applicant: TARVEDA THERAPEUTICS, INC., Watertown, MA (US)

(72) Inventors: Sudhakar Kadiyala, Newton, MA (US); Mark T. Bilodeau, Waltham, MA (US); Donna T. Ward, Groton, MA (US)

(21) Appl. No.: 15/762,627

(22) PCT Filed: Sep. 23, 2016
(86) PCT No.: PCT/US2016/053325

§ 371 Date: Mar. 23, 2018

Related U.S. Application Data
(60) Provisional application No. 62/232,625, filed on Sep. 25, 2015.

(51) Int. Cl. C12N 9/22; C12N 15/11; C12N 15/90

(52) U.S. Cl. CPC C12N 9/22; C12N 15/11; C12N 2320/32; C12N 2310/20; C12N 15/907

(57) ABSTRACT

The present invention provides conjugates, nanoparticles and compositions comprising components of a CRISPR-Cas system; these compositions can be used for genetic editing in a cell or an organism.

### REFERENCE TO RELATED APPLICATIONS

The present application claims priority to U.S. Provisional Patent Application No. 62/232,625, filed Sep. 25, 2015, entitled Compositions and methods for genome editing, the contents of each of which are herein incorporated by reference in its entirety.

### FIELD OF THE INVENTION

The present invention provides compositions for genetic editing in a cell using the CRISPR-Cas system.

### BACKGROUND OF THE INVENTION

An ease and targeted Genome editing has been honed by scientists for many aspects of biological and medical researches. Various methods and compositions for targeted cleavage of genomic DNA have been described. Generally these methods involve the use of engineered cleavage systems to induce a double strand break (DSB) or a nick in a target DNA sequence such that repair of the break by non-homologous end joining (NHEJ) or repair using a repair template (homology directed repair or HDR) can result in the knock out of a gene or the insertion of a sequence of interest. Cleavage can occur through the use of specific nucleases such as engineered zinc finger nucleases (ZFN), transcription-activator like effector nucleases (TALENs) and nucleases based on the Argonaute system (e.g., from T. thermophilus, known as ‘TtAgo’, (Swarts et at (2014) Nature 507 (7491): 258-261).

The CRISPR-Cas9 system is a novel genome editing system which has been rapidly developed and implemented in a multitude of model organisms and cell types, and supplants other genome editing technologies, such as TALENs and ZFNs. CRISPRs are sequence motifs are present in bacterial and archaeal genomes, and are composed of short (about 24-48 nucleotide) direct repeats separated by similarly sized, unique spacers (Grissa et al. BMC Bioinformatics 8, 172 (2007)). They are generally flanked by a set of CRISPR-associated (Cas) protein-coding genes that are required for CRISPR maintenance and function (Barrangou et al., Science 315, 1709 (2007), Brouns et al., Science 321, 960 (2008), Haft et al. PLoS Comput Biol 1, e60 (2005)). CRISPR-Cas systems provide adaptive immunity against invasive genetic elements (e.g., viruses, phages and plasmids) (Horvath and Barrangou, Science, 2010, 327: 167-170; Bhaya et al., Annu. Rev. Genet., 2011, 45: 273-297; and Brrangou R, RNA, 2013, 4: 267-278). Three different types of CRISPR-Cas systems have been classified in bacteria and the type II CRISPR-Cas system is most studied. In the bacterial Type II CRISPR-Cas system, small CRISPR RNAs (crRNAs) processed from the pre-repeat-spacer transcript (pre-crRNA) in the presence of a trans-activating RNA (tracrRNA)/Cas9 can form a duplex with the tracrRNA/Cas9 complex. The mature complex are recruited to a target double strand DNA sequence that is complementary to the spacer sequence in the tracrRNA:crRNA duplex to cleave the target DNA by Cas9 endonuclease (Garneau et al., Nature, 2010, 468: 67-71; Jinek et al., Science, 2012, 337: 816-821; Gasiunas et al., Proc. Natl Acad. Sci. USA., 109: E2579-2586; and Haurwitz et al., Science, 2010, 329: 1355-1358). Target recognition and cleavage by the crRNA:tracrRNA/Cas9 complex in the type II CRISPR-CAS system not only requires a sequence in the tracrRNA:crRNA duplex that is a complementary to the target sequence (also called “protospacer” sequence) but also requires a protospacer adjacent motif (PAM) sequence located 3′end of the protospacer sequence of a target polynucleotide. The PAM motif can vary between different CRISPR-Cas systems.

CRISPR-Cas9 systems have been developed and modified for use in genetic editing and prove to be a high effective and specific technology for editing a nucleic acid sequence even in eukaryotic cells. Many researchers disclosed various modifications to the bacterial CRISPR-Cas systems and demonstrated that CRISPR-Cas systems can be used to manipulate a nucleic acid in a cell, such as in a mammalian cell and in a plant cell. Representative references include U.S. Pat. Nos. 8,993,233; 8,999,641; 8,945,839; 8,932,814; 8,906, 616; 8,895,308; 8,889,418; 8,889,356; 8,871,445; 8,865,406; 8,771,945; and 8,697,359; US patent publication NOs.: 20150031134; 20150203872; 20150218253; 20150176013; 20150191744; 20150071889; 20150067922; and 20150167000; each of which is incorporated herein by reference in their entirety.

However, delivering components of the CRISPR-Cas system (e.g., guide RNA and nuclease) has been challenging and often can be problemic. For example, delivery of a nuclease via transduction of a plasmid into the cell can be toxic to the recipient cell. As provided herein the present invention provides a novel conjugate and nanoparticle for increased targeting of CRISPR-Cas9 system to a particular tissue or cell of interest and increases the efficacy of genome editing.

### SUMMARY OF THE INVENTION

In one aspect of the present invention, conjugates, nanoparticles and formulations comprising components of CRISPR-Cas system are provided. Conjugates comprise guide RNA molecules as active agents which are connected the targeting moiety through the linker. In some embodiments, a Cas protein, together with conjugates comprising guide RNAs, is packaged to nanoparticles and/or formulations of the present invention. The present compositions comprising components of a CRISPR-Cas system may be used for genetic editing in a cell, such as a mammalian cell.

In one embodiment of the present invention, the CRISPR-Cas system is a Type II CRISPR-Cas system and the Cas protein is Cas9 which catalyzes DNA cleavage.

### DETAILED DESCRIPTION OF THE INVENTION

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

### Definitions

The terms used in this invention are, in general, expected to adhere to standard definitions generally accepted by those having ordinary skill in the relevant art.

CRISPR-Cas system: As used herein, the term “CRISPR-Cas system” in general refers collectively to components/elements involved in directing the activity of CRISPR-associated (“Cas”) proteins, including sequences encoding a Cas gene, a tracr (trans-activating CRISPR) sequence (e.g. tracrRNA or an active partial tracrRNA), a tracr-complementary sequence (encompassing a “direct repeat” and a tracrRNA-processed partial direct repeat in the context of an endogenous CRISPR system), a target (guide) sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR-Cas system. In some embodiments, one or more elements of a CRISPR-Cas system is derived from a particular organism which may comprise an endogenous CRISPR-Cas system, such as Streptococcus pyogenes. In general, a CRISPR-Cas system is characterized by components that promote the formation of a CRISPR/Cas complex at the site of a target sequence (also referred to as a protospacer in the context of an endogenous CRISPR-Cas system). In the context of formation of a CRISPR complex, “target sequence” refers to a sequence to which a target recognition sequence is designed to have complementarity, where hybridization between a target sequence and a target recognition sequence promotes the formation of a CRISPR/Cas complex. A target sequence may comprise any polynucleotide, such as DNA or RNA polynucleotides. In some embodiments, a target sequence is located in the nucleus or cytoplasm of a cell.

CRISPR interference (CRISPRi): As used herein, the term “CRISPRi” refers to a genetic perturbation technique that allows for sequence-specific repression or activation of gene expression in prokaryotic and eukaryotic cells using CRISPR complexes. CRISPRi regulates gene expression primarily on the transcriptional level.

Compound: As used herein, the term ““compound”, as used herein, is meant to include all stereoisomers, geometric isomers, tautomers, and isotopes of the structures depicted. In the present application, compound is used interchangeably with conjugate. Therefore, conjugate, as used herein, is also meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Complementary: As used herein, the term “complementary” or “complementarity” are used in reference to polynucleotides related by the base-pairing rules. For example, the sequence of “C-T-G-A” is complementary to the sequence of “G-A-C-T”. Complementarity can be total or partial. Partial complementarity is where one or more nucleic acid base is not matched according to the base-pairing rules. Total or complete complementarity between polynucleotides is where each and every nucleic acid base is matched with another base under the base pairing rules.

Copolymer: As used herein, the term “copolymer” generally refers to a single polymeric material that is comprised of two or more different monomers. The copolymer can be of any form, such as random, block, graft, etc. The copolymers can have any end-group, including capped or acid end groups.

Domain: As used herein, the term “domain” or “protein domain” refers to a part of a protein sequence that may exist and function independently of the rest of the protein chain.

Duplex: As used herein, the term “duplex” describes two complementary polynucleotides that are base-paired, i.e., hybridized together, for example, a tracrRNA:crRNA duplex.

Editing: As used herein, the term “editing”, “edit”, “edition”, or “edited” refers to a method of altering a nucleic acid sequence of a polynucleotide (e.g., a naturally-occurring wild type nucleic acid sequence or a naturally-occurring mutated nucleic acid sequence by introducing a change to a specific genomic target; the genomic target may include a chromosomal region, a coding polynucleotide (e.g., a gene), a promotor, a non-coding polynucleotide, or any nucleic acid sequence. The changes to a nucleic acid may include deletion, addition and other changes to the nucleic acid sequence in the genome.

Encode: as used herein, the term “encode”, or “encoding” or “encoded” refers to a nucleic acid sequence that codes for a polypeptide sequence.

Genome: As used herein, the term “genome” means the complete genetic information present in a cell or organism.

Guide RNA (gRNA): As used herein, the term “guide RNA” refers to a RNA molecule used in conjunction with a CRISPR associated system. The guide RNA may be composed of two RNA molecules, i.e., one RNA (“crRNA”) which hybridizes to a target sequence and provides sequence specificity, and one RNA, the “tracrRNA”, which is capable of hybridizing to the crRNA and forming a duplex with crRNA upon hybridization. In some embodiments the guide RNA may be a single guide RNA (sgRNA). sgRNA contains nucleotide sequence specific to a non-variable scaffold sequence of the 5′ end of a target DNA (i.e. crRNA) and tracrRNA sequence. SgRNA can be delivered as RNA or by transforming with a plasmid with sgRNA coding sequence under a promotor sequence. The base pairing of sgRNA with the target sequence recruits a Cas protein (e.g., the Cas9 nuclease) to bind the DNA at that locus and cleave the target DNA sequence. As used herein, the term “crRNA” is intended to refer to the endogenous bacterial RNA that confers target specificity, which requires tracrRNA to bind to Cas9. As used herein, the term “tracrRNA” refers to the endogenous bacterial RNA that links the crRNA to the Cas9 nuclease and can bind any crRNA.

Hybridization: As used herein, the term “hybridize” or “Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

Peptide, polypeptide and protein: As used herein, the terms “peptide”, “polypeptide” and ‘protein” are used interchangeably to refer to a series of amino acid residues joined by peptide bonds (i.e., a polymer of amino acids). The amino acid residues include naturally-occurring 21 alpha amino acids and modified amino acids (e.g., phosphorylated, glycated, glycosolated, etc.) and amino acid analogs. Exemplary polypeptides or proteins include gene products, naturally occurring proteins, homologs, paralogs, fragments and other equivalents, variants, and analogs of the above. These terms include post-transcription modifications of the polypeptide, for example, glycosylations, acetylations, phosphorylations and the like.

Nucleic acid: As used herein, the term “nucleic acid,” as well as the terms “polynucleotide,” and “oligonucleotide” are used interchangeably and refer to a deoxyribonucleotide (DNA) or ribonucleotide (RNA) polymer, in linear or circular conformation, and in either single- or double-stranded form. For the purposes of the present disclosure, these terms are not to be construed as limiting with respect to the length of a polymer. The length of a nucleic acid molecule may be greater than about 2 bases, greater than about 10 bases, greater than about 100 bases, greater than about 500 bases, greater than 1000 bases, up to about 10,000 or more bases composed of nucleotides. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T. A nucleic acid molecule may be produced enzymatically or synthetically.

Target polynucleotide: as used herein, the term “target polynucleotide” refers to a polynucleotide of interest under study. In certain embodiments, a target polynucleotide contains one or more sequences that are of interest and under study.

Vector: As used herein, the term “vector” refers to a replicon to which another polynucleotide segment is attached. A vector is used to bring about the transcription, replication and/or expression of the attached polynucleotide segment. As such, the vector can include origin of replications, promoters, multicloning sites, selectable markers and combinations thereof. Vectors can include, for example, plasmids, viral vectors, cosmids, and artificial chromosomes.

### Compositions of the Invention

Compositions of the present inventions include conjugates comprising a targeting moiety, a linker, and one or more active agents, e.g., one or more guide RNAs that may conjugated to the targeting moiety through a linker. Nanoparticles that package one or more conjugates of the present invention are also provided. The conjugates can be encapsulated into nanoparticles or disposed on the surface of the nanoparticles. In particular, conjugates of the present invention and nanoparticles comprising such conjugates may be used as programmable genetic editing tools. The conjugates, nanoparticles comprising the conjugates, and/or formulations thereof can provide improved temporospatial delivery of the active agent (e.g., sgRNAs for a target sequence of interest and a Cas9 enzyme or its variants) and/or improved biodistribution compared to delivery of the active agent alone.

### I. The CRISPR-Cas System and CRISPR Technology

The CRISPR (Clustered Regularly Interspaced Short Palidromic Repeats)-Cas system is widely found in bacterial and archaeal genomes as a defense mechanism against invading viruses and mobile genetic elements such as bacteriophages and plasmids, which sometimes is called RNA-mediated adaptive immune system. A CRISPR locus in a bacterial genome is a DNA region with an array of short identically repeated sequences of generally 21-37 base pairs, separated by spacers with unique sequences of generally 20-40 base pairs. The CRISPR locus can be found on both chromosomal and plasmid DNA. The spacers are often derived from nucleic acid of invading viruses and plasmids, which can be used as recognition elements to find matching virus genomes or plasmid sequences and destroy them as part of defense system. CRISPR activity requires the presence of a set of CRISPR associated (cas) genes, which are usually found adjacent to the CRISPR array and encode CRISPR associated (Cas) proteins with a variety of predicted nucleic acid-manipulating activities such as nucleases, helicases and polymerases. The CRISPR-Cas system targets DNA and/or RNA as a way of protecting against viruses and other mobile genetic elements, and can be developed for programmable genetic editing.

In bacteria, the CRISPR-Cas system relies on the activity of short mature CRISPR RNAs (crRNAs) that guide Cas proteins (e.g. Cas9) to silence invading nucleic acids. The crRNRs are processed from the DNA sequences clustered in the CRISPR array. The CRISPR array transcript, the precursor CRISPR RNA (pre-crRNA), is processed into individual short mature crRNAs. In the CRISPR-Cas type II system, a small, non-coding RNA molecule called trans-activating crRNA (tracrRNA), which has a sequence complementary to the identical repeat in the CRISPR, is required for crRNA maturation. The tracrRNA is encoded in the vicinity of the cas genes and CRISPR repeat-spacer array. Following the hybridization of tracrRNA to the short identical repeat in the pre-crRNA, the bacterial double-stranded RNA specific endoribonuclease, RNase III, processes/cleaves the pre-crRNA transcript to generate a dual-tracrRNA:crRNA that guides the CRISPR-associated endonuclease Cas9 (Csn1) to cleave site-specifically cognate target DNA.

In bacteria, CRISPR-Cas immunity operates in three steps with the principle that an intruder once memorized by the system will be remembered and silenced upon a repeated infection. During the initial adaptation phase, a part of an invading nucleic acid sequence is incorporated as a new spacer within the repeat-spacer CRISPR array and the infection is thus memorized. During the expression phase, the CRISPR array is transcribed as a pre-crRNA molecule that undergoes processing to generate short mature crRNAs, each complementary to a unique invader sequence. During the interference phase, the individual crRNAs guide Cas protein(s) to cleave the cognate invading nucleic acids in a sequence-specific manner for their ultimate destruction.

The CRISPR-Cas systems have recently been classified into three distinct types (I-III). Types I and III share some common features, with crRNAs and Cas proteins being the only known components required for the steps of expression and interference. In both types I and III, the mature crRNAs guide a complex of several Cas proteins to the cognate-invading nucleic acids and a Cas endonuclease of the ribonucleoprotein complex cleaves the target nucleic acids. Type II CRISPR-Cas has evolved distinct pre-crRNA processing and interference mechanisms, as described above. Pre-crRNA processing requires base-pairing of every pre-crRNA repeat with a tracrRNA. The tracrRNA: crRNA duplex forms a ternary silencing complex in the presence of Cas9, the endonuclease of Type II CRISPR-Cas system.

Though naturally being identified as a defense mechanism in prokaryotes, the CRISPR-Cas system (e.g., the Type II CRISPR-Cas9 system) has been developed as a RNA-guided DNA targeting platform. It has been widely studied for the use of genomic editing and transcription modulation in eukaryotic cells, and has shown great potential in correcting mutations in human genetic diseases. As learned from the bacterial system, two distinct components are required in CRISPR-Cas based genome editing: (1) a guide RNA molecule, such as a tracrRNA:crRNA duplex and (2) an endonuclease, such as Cas9 or Cpf1. When the guide RNA molecule and Cas9 are expressed in a cell, the guide RNA/Cas9 complex is recruited to the target sequence by the base-pairing between the guide RNA sequence and the target sequence in the genomic DNA. The recruited Cas9 cuts both strands of DNA causing a Double Strand Break (DSB). For successful location of Cas9 to the target sequence, the genomic target sequence must also contain the correct Protospacer Adjacent Motif (PAM) sequence immediately following the target sequence. Cas9 cuts 3-4 nucleotides upstream of the PAM sequence. A DSB can be repaired through one of two general repair pathways: (1) the Non-Homologous End Joining (NHEJ) DNA repair pathway, or (2) the Homology Directed Repair (HDR) pathway. The NHEJ repair pathway often results in inserts/deletions at the DSB site that can lead to frameshifts and/or premature stop codons, effectively disrupting the open reading frame (ORF) of the targeted gene. The HDR pathway requires the presence of a repair template, which is used to fix the DSB. HDR faithfully copies the sequence of the repair template to the cut target sequence. Specific nucleotide changes can be introduced into a targeted gene by the use of HDR with a repair template.

The CRISPR-Cas system has been used as a tool to manipulate the genome in mammalian cells (i.e. eukaryotes). Wu et al (Wu et al., Science, 2013, 339: 819-823) and Mali et al (Mali et al., Science. 2013, 339: 823-826) first demonstrated that expressing a codon-optimized Cas9 protein and a guide RNA leads to efficient cleavage and short insertion/deletion of target loci, which could inactivate protein-coding genes by inducing frameshifts and/or creating premature stop codons. The CRISPR-cas system can be used to simultaneously edit more than one genes by delivering multiple guide RNAs (Yang et al., Cell, 2013, 154:1370-1379; and Jao et al., Proc Natl Acad Sci USA. 2013, 110:13904-13909); to introduce deletions and inversions of regions range from 100 bps to 1000000 bps on a chromosome (Xiao et al., Nucleic Acids Res., 2013, 41:e141; and Canver et al., J Biol Chem., 2014, 289(31): 21312-21324); to engineer chromosomal translocations between different chromosomes (Torres et al., Nat Commun. 2014, 5: 3964); to correct mutations in disease genes (Yin et al., Nat Biotechnol. 2014, 32(6): 551-553; Schwank et al., Cell Stem Cell. 2013, 13: 653-658; and Wu et al., Cell Stem Cell. 2013, 13: 659-662); and to introduce specific nucleotide modifications at the target sequence in combination with a DNA repair template containing the desired sequence but having a high degree of homology to the target sequence, for example, by cotransfection of the template DNA into the cell along with the guide RNA/Cas9 complex.

The CRISPR-Cas system has also been adapted to label proteins by introducing specific sequences such as HA-tag (Auer et al., Genome Res. 2014; 24:142-153). Other expended applications of the CRISPR-Cas system include site-specific imaging of endogenous loci in living cells, by using a fluorescent marker (e.g., GFP). In addition to human and rodent cells, the system has also been adapted to many other species, including monkey, pig, rat, zebrafish, worm, yeast, and several plants.

The CRISPR-Cas system is a remarkably flexible tool for genome manipulation. One of the primary advantages of this technology is that the nuclease activity and the DNA-binding activity of Cas9 are discrete functions in the protein. The Cas9 nuclease activity (cutting) is performed by 2 separate domains, RuvC and HNH. Each domain cuts one strand of DNA and each can be inactivated by a single point mutation. In S. pyogenes, a Cas9 D10A mutant has an inactive RuvC domain (RuvC−) and an active HNH domain (HNH+) and a Cas9 H840A mutant has an inactive HNH domain (HNH−) and an active RuvC domain (RuvC+). When both domains are inactive (D10A and H840A, RuvC− and HNH−) the Cas9 protein has no nuclease activity (catalytically inactive) and is said to be ‘dead’ (dCas9); however, the inactive dCas9 still retains the ability to bind to DNA based on guide RNA specificity. Such dCas9 protein may be used as a platform to recruit other functional proteins to a target DNA sequence.

The CRISPR-cas system may also be used to modulate transcription in a genome by introducing sequence specific control of gene expression. In some applications, a catalytically inactive dCas9 can be generated by mutating the two nuclease domains of Cas9, which can bind DNA without introducing cleavage or mutation. When targeted to a promoter, the nuclease-null dCas9 binding alone can interfere with transcription initiation, likely by blocking binding of transcription factors or RNA polymerases. When targeted to the non-template strand within the gene, the dCas9 complex blocks RNA polymerase II transcription elongation (Jinek et al., Science. 2012, 337(6096): 816-821; Qi et al., Cell. 2013, 152:1173-1183; and Gilbert et al., Cell. 2013, 154: 442-451). In other applications, the inactive or nuclease-null cas9 may be fused with effector domains with distinct regulatory functions, such as transcription repressor domains (e.g., the Krueppel-associated box (KRAB)) to lead to stronger silencing of mammalian genes (Gilbert et al., Cell, 2013, 154(2): 442-451), or with activator domains (e.g., VP64) to activate transcriptions (Larson et al., Nat Protoc. 2013, 8: 2180-2196). Such guide RNA based methods are referred to as CRISPR interference (i.e. CRISPRi) (Qi et al., Cell, 2013, 152: 1173-1183; and Larson et al., Nature Protocols, 2013, 8: 2180-2196). Such RNA based methods for regulation of gene expression in a genome-wide scale are also known as CRISPR interference ((i.e., CRISPR interference, CRISPRi).

In addition to modulating gene expression, the fusion of dCas9 with other heterologous effector domains could enable many other applications. For example, one could fuse catalytically inactive dCas9 with chromatin modifiers to change the epigenetic state of a locus (Mali et al., Nat Methods. 2013, 10:957-963; and Sander et al., Nat Biotechnol. 2014, 32: 347-355). dCas9 fused to an epitope tag(s) can be used to purify genomic DNA bound by the guide RNA. Building on the well-established concept of ChIP (Chromatin Immunoprecipitation), researchers have created enChIP (engineered DNA-binding molecule-mediated ChIP) that allows for the purification of any genomic sequence specified by a particular guide RNA. By creating pooled libraries of guide RNAs, the CRISPR-Cas system can be used in powerful genomic screening techniques or for nucleic acid enrichment (See, e.g., US patent publication NO.: 20140356867).

### A. The Cas Proteins

Currently the most commonly used RNA-guided endonuclease for genome editing in the CRISPR-Cas system is the Type II CRISPR associated (Cas) nuclease, Cas9. The Cas9 nuclease is a DNA endonuclease with two nuclease domains, namely, the N-terminal RuvC-like nuclease (RNAse H fold) and the HNH (McrA-like) nuclease domain that is located in the middle of the protein, each cleaving each of the two DNA strands. When both of these domains are active, the Cas9 protein causes double strand breaks (DSBs) in the genomic DNA. In the absence of a suitable repair template, the DSB is repaired by the Non-Homologous End Joining (NHEJ) DNA repair pathway. During NHEJ repair, InDels (insertions/deletions) may occur as a small number of nucleotides are either inserted or deleted at random at the DSB site.

Cas9 may be inactivated with both the functional domains mutated ((RuvC− and HNH−), generating a nuclease-null Cas9 (dCas9). Cas9 may also be modified as “nickase: a Cas9 protein containing a single inactive catalytic domain, either RuvC− or HNH−. With only one active nuclease domain, the Cas9 nickase cuts only one strand of the target DNA, creating a single-strand break or ‘nick’. Similar to the inactive dCas9, a Cas9 nickase is still able to bind DNA based on guide RNA specificity, though nickases will only cut one of the DNA strands. A single-strand break, or nick, is normally quickly repaired through the HDR pathway, using the intact complementary DNA strand as the template. Two proximal, opposite strand nicks introduced by a Cas9 nickase (often referred to as a ‘double nick’ or ‘dual nickase’ CRISPR system) are treated as a Double Strand Break (DSB), which can be repaired by either NHEJ or HDR depending on the desired effect on the gene target.

The most commonly used Cas9 is derived from Streptococcus pyogenes and the RuvC domain can be inactivated by a D10A mutation and the HNH domain can be inactivated by an H840A mutation.

In addition to Cas9 derived from S. pyogenes, other RNA guided endonucleases (RGEN) may also be used for programmable genome editing. Cas9 sequences have been identified in more than 600 bacterial strains. Though Cas9 family shows high diversity of amino acid sequences and protein sizes, All Cas9 proteins share a common architecture with a central HNH nuclease domain and a split RuvC/RHase H domain. Examples of Cas9 orthologs from other bacterial strains including but not limited to, Cas proteins identified in Acaryochloris marina MBIC11017; Acetohalobium arabaticum DSM 5501; Acidithiobacillus caldus; Acidithiobacillus ferrooxidans ATCC 23270; Alicyclobacillus acidocaldarius LAA1; Alicyclobacillus acidocaldarius subsp. acidocaldarius DSM 446; Allochromatium vinosum DSM 180; Ammonifex degensii KC4; Anabaena variabilis ATCC 29413; Arthrospira maxima CS-328; Arthrospira platensis str. Paraca; Arthrospira sp. PCC 8005; Bacillus pseudomycoides DSM 12442; Bacillus selenitireducens MLS10; Burkholderiales bacterium 1_1_47; Caldicelulosiruptor becscii DSM 6725; Candidatus Desulforudis audaxviator MP104C; Caldicellulosiruptor hydrothermalis_108; Clostridium phage c-st; Clostridium botulinum A3 str. Loch Maree; Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB17; Methanohalobium evestigatum Z-7303; Microcystis phage Ma-LMM01; Microcystis aeruginosa NIES-843; Microscilla marina ATCC 23134; Microcoleus chthonoplastes PCC 7420; Neisseria meningitidis; Nitrosococcus halophilus Nc4; Nocardiopsis dassonvillei subsp. dassonvillei DSM 43111; Nodularia spumigena CCY9414; Nostoc sp. PCC 7120; Oscillatoria sp. PCC 6506; Pelotomaculum thermopropionicum SI; Petrotoga mobilis SJ95; Polaromonas naphthalenivorans CJ2; Polaromonas sp. J5666; Pseudoalteromonas haloplanktis TAC125; Streptomyces pristinaespiralis ATCC 25486; Streptomyces pristinaespiralis ATCC 25486; Streptococcus thermophilus; Streptomyces viridochromogenes DSM 40736; Streptosporangium roseum DSM 43021; Synechococcus sp. PCC 7335; and Thermosipho africanus TCF52B (Chylinski et al., RNA Biol., 2013; 10(5): 726-737).

In addition to Cas9 orthologs, other Cas9 variants such as fusion proteins of inactive dCas9 and effector domains with different functions may be served as a platform for genetic modulation.

Other than CRISPR/Cas9 system, Clustered Regularly Interspaced Short Palindromic Repeats from Prevotella and Francisella 1 (CRISPR/Cpf1) is analogous to the CRISPR/Cas9 system. Cpf1 is an RNA-guided endonuclease of a class II CRISPR/Cas system. Cpf1 is smaller than Cas9 in size.

### B. The Guide RNA

In the CRISPR-Cas technology, a RNA molecule is required to guide the Cas9 endonuclease to site-specifically cleave target DNA determined by the PAM motif. In most applications, a chimeric single guide RNA (aka sgRNA) that combines the targeting specificity of the crRNA with the scaffolding properties of the tracrRNA for Cas9 into a single transcript, is used. In some examples, the sgRNA is a combination of the endogenous bacterial crRNA and tracrRNA.

A sgRNA molecule generally includes a target recognition sequence that can hybridize to a 20-nucleotide DNA sequence of the genomic DNA that immediately precedes the PAM (Protospacer Adjacent Motif) sequence recognized by Cas9 (e.g., a NGG motif). Thus, the target DNA sequence, for example may be a (N)₂₀NGG target DNA sequence. The 20 complementary nucleotides are put into a sgRNA molecule, but not the PAM sequence. The PAM is a required sequence that must immediately follow the sgRNA recognition sequence but is not included in the sgRNA molecule, such as a sgRNA plasmid.

Target sequences (20 nucleotides+PAM) can be on either strand of the genomic DNA. Target sequences can appear in multiple places in the genome. Any DNA sequence with the correct target sequence followed by the PAM sequence will be bound by the Cas9 nuclease. The PAM sequence varies by the species of the bacteria from which the Cas9 was derived. The most widely used Type II CRISPR-Cas system is derived from S. pyogenes and the PAM sequence is NGG located on the immediate 3′ end of the sgRNA recognition sequence.

The PAM sequences of other Type II CRISPR systems from different bacterial species are listed in the Table below.

TABLE 1
PAM motifs

| Species | PAM sequences |
|---|---|
| Streptococcus pyogenes (SP) | NGG |
| Staphylococcus aureus (SA) | NNGRRT (or NNGRR(N)) |
| Neisseria meningitidis (NM) | NNNNGATT |
| Streptococcus thermophilus | NNAGAAW |
| (ST) | |
| Treponema denticola (TD) | NAAAAC |

In addition to the recognition sequence that determines the specificity of a DNA target, a sgRNA contains a sequence to which Cas9 endonuclease binds. The Cas9 binding sequence may be 30 to 40 nucleotides in length.

Alternatively, the target recognition sequence and the Cas9 binding sequence can be introduced into a cell along with the Cas9 protein as separate RNA molecules. For example, a tracrRNA and a crRNA may be introduced into a cell together with the Cas9 protein, In this application, the tracrRNA and crRNA form a crRNA:tracrRNA duplex to direct Cas9 to and hybridize to a target motif of the target polynucleotide sequence.

The sgRNAs may be a small number of sgRNAs for a gene of interest, or an entire library of sgRNAs to cover a genome. The sgRNA sequences may be designed to include features that enhance on-target activity of sgRNAs and can avoid the off-target activity of sgRNAs. A gRNA can be designed with increased targeting specificity using a number of tools available to help choose/design target sequences as well as lists of bioinformatically determined unique gRNAs for different genes in different species, such as Target Finder from Dr. Feng Zhang, E-CRISP (Heigwer et al., Nature Methods, 2014; 11: 122-123), Cas-OFFinder (Bae et al., Bioinformatics, 2014, 30:1473-1475), CasFinder (Aach et al., BioRxiv, doi: http://dx.doi.org/10.1101/005074).

### II. Conjugate of the Invention

In accordance with the present invention, conjugates comprise at least three moieties: a targeting moiety (or ligand), a linker, and an active agent called a payload that is connected to the targeting moiety via the linker. In some embodiments, the conjugate may be a conjugate between a single active agent and a single targeting moiety with the formula (I): X—Y—Z, wherein X is the targeting moiety; Y is a linker; and Z is the active agent. In certain embodiments, one targeting ligand can be conjugated to two or more payloads wherein the conjugate has the formula: X—(Y—Z)ₙ. In certain embodiments, one active payload can be linked to two or more targeting ligands wherein the conjugate has the formula: (X-Y)ₙ—Z. In other embodiments, one or more targeting ligands may be connected to one or more active payloads wherein the conjugate formula may be (X—Y—Z)ₙ. In various combinations, the formula of the conjugates maybe, for example, X—Y—Z—Y—X, (X—Y—Z)ₙ—Y—Z, or X—Y—(X—Y—Z)ₙ, wherein X is a targeting moiety; Y is a linker; Z is an active agent. The number of each moiety in the conjugate may vary dependent on types of agents, sizes of the conjugate, delivery targets, particles used to packaging the conjugate, other active agents (e.g., immunologic adjuvants) and routes of administration. Each occurrence of X, Y, and Z can be the same or different, e.g. the conjugate can contain more than one type of targeting moiety, more than one type of linker, and/or more than one type of active agent. n is an integer equal to or greater than 1. In some embodiments, n is an integer between 1 and 50, or between 2 and 20, or between 5 and 40. In some embodiments, n may be an integer of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 41, 43, 44, 45, 46, 47, 48, 49 or 50.

### A. Payloads

As used herein, the terms “payload” and “active agent” are used interchangeably. A payload may be any active agents such as therapeutic agents, prophylactic agents, or diagnostic/prognostic agents. A payload may have a capability of manipulating a physiological function (e.g., gene expression) in a subject. One payload may be included in the present conjugate. One or more, either the same or different payloads may be included in the present conjugate.

In accordance with the present invention, a payload or an active agent may be guide RNAs (gRNAs) associated with a CRISPR-Cas system. In some embodiments, the guide RNA is a single guide RNA (sgRNA) that is used as a component of the CRISPR-Cas system. In one aspect, the sgRNA is a synthetic and chimeric sgRNA.

In some embodiments, the guide RNA molecule (gRNA or sgRNA) comprises a target recognition sequence that specifically recognizes and hybridizes to a target sequence (i.e. a motif) of a target polynucleotide, a tracr sequence and a sequence complementary to the tracr sequence. The target motif refers to about 20 nucleotides in length within the target polynucleotide and immediately precedes a PAM motif recognized by the Cas9 protein. As a non-limiting example, a target motif may be (N)₂₀NGG. As used herein, the sequence complementary to the tracr sequence is equal to the “repeat” sequence in the CRISPR array. In general, the target recognition sequence may be any polynucleotide sequence having sufficient complementarity with a target polynucleotide sequence to hybridize with the target sequence and direct sequence-specific binding of a CRISPR-Cas complex to the target sequence. In some embodiments, the degree of complementarity between a target recognition sequence of a sgRNA molecule and its corresponding target sequence, when optimally aligned using a suitable alignment algorithm (e.g., Waterman algorithm, the Needleman-Wunsch algorithm, the Burrows Wheeler Aligner, ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net)), is about or more than about 50%, 60%, 75%, 80%, 85%, 90%, 95%, 97.5%, 99%, or more.

In some embodiments, the target recognition sequence of a sgRNA molecule comprises 12-25 nucleotides that are complementary to and/or hybridize to the 12-25 consecutive nucleotides of the target motif of a selected polynucleotide in a genome. The target recognition sequence may be 12, 13, 14, 15, 16, 17, 18, 19, 20, 21, 22, 23, 24, 25 nucleotides in length. In one particular example, a target recognition sequence of a sgRNA molecule may comprise 20 nucleotide. In other embodiments, the target recognition sequence of a sgRNA molecule may comprise about 20, to about 100 nucleotides, such as about 25, 30, 35, 40, 45, 50, 60, 70, 80, 90, 100 nucleotides in length. In other embodiments, the target recognition sequence of a sgRNA may be fewer than 20, 18, 16, 15 or 12 nucleotides in length. The ability of a target recognition sequence to direct sequence-specific binding of a CRISPR-Cas complex to a target sequence may be assessed by any suitable assay.

In some embodiments, the target recognition sequence of a sgRNA may be selected and designed to reduce the degree of secondary structure within the sgRNA molecule. In some embodiments, about or less than about 70%, 50%, 40%, 30%, 25%, 20%, 15%, 10%, 5%, 1%, or fewer of the nucleotides of the target recognition sequence participate in self-complementary base pairing when optimally which can be determined by any suitable polynucleotide folding algorithm (e.g., mFold).

The target sequence may be any polynucleotide sequence in a genome of a cell or an organism of interest. The target sequence may be unique in the genome (i.e., only one copy); or repeat sequences in the genome. For example, if the Cas9 protein from S. pyogenes is used, a target sequence in the genome may include the sequence of XGG (N represents A, G, T, and C; and X can be anything). In the case that the CRISPRI Cas9 protein from S. thermophiles is used, a target sequence may include XXAGAAW (N is A, G, T, C; X can be anything and W is A or T).

In some examples, the target recognition sequence of the sgRNA molecule may be joined to the tracr sequence. In other examples, the tracr sequence may be truncated at various positions. The guide and tracr sequences are separated by the sequence complementary to the tracr sequence. In other examples, the sequence complementary to the tracr sequence may be further followed by a loop sequence, such as GAAA.

In some embodiments, the tracr sequence may comprise or consist of all or a portion of a wild-type tracr sequence. In some examples, the tracr sequence may be about or more than about 20, 22, 24, 26, 28, 30, 32, 40, 45, 50, 55, 60, 65, 70, 75, 85, or more nucleotides of a wild-type tracr sequence, or derivatives thereof. The tracr sequence may also form part of a CRISPR-Cas complex, such as by hybridization along at least a portion of the tracr sequence to all or a portion of a sequence complementary to the tracr sequence that is operably linked to the target recognition sequence.

In some embodiments, the sequence complementary to the tracr sequence with the sgRNA molecule may be any sequence that has sufficient complementarity with a tracr sequence. The degree of complementarity between a tracr sequence and its complementary sequence along the length of the shorter of the two when optimally aligned may be about or more than about 25%, 30%, 40%, 50%, 60%, 70%, 80%, 90%, 95%, 97.5%, 99%, or higher. Optimal alignment may be determined by any suitable alignment algorithm as discussed above. The sequence complementary to the tracr sequence may promote the excision of a target recognition sequence flanked by tracr complementary sequences in a cell containing the corresponding tracr sequence; and the formation of a CRISPR-Cas complex at a target sequence.

In some embodiments, the sgRNA may be complexed with one or more Cas proteins. In one example, the sgRNA may be complexed with the Cas9 endonuclease.

In some embodiments, the sgRNA molecule may be chemically modified to increase its stability and/or to enhance Cas9 association; and/or reduce off-target effects for in vivo application. The RNA can be modified in the nucleobase structure or in the ribose-phosphate backbone structure. The modifications may be similar to modifications discussed in the following sections of the present application. Such modifications retain all the activity of an unmodified sgRNA. As non-limiting examples, a sgRNA molecule may include at least one modified ribonucleoside selected from the group consisting of pseudouridine, 5-methylcytodine, 2-thiouridine, 5-methyluridine-5′-triphosphate, 4-thiouridine-5′-triphosphate, 5,6-dihydrouridine-5′-triphosphate, and 5-azauridine-5′-triphosphate.

In some embodiments, more than one sgRNA molecules may be connected to a targeting moiety through the linker. In some examples, two, three, four, five or multiple sgRNAs are included in the present conjugate. The multiple sgRNAs molecules may comprise different target recognition sequences derived from different target motifs of the same selected polynucleotide in a genome, or alternatively may comprise different target recognition sequences derived from different target motifs of different selected target polynucleotides in a genome, such as two, three, four, or multiple different polynucleotides.

In some embodiments, the sgRNA may be a truncated sgRNA (tru-sgRNA), which is deleted at the 5′ end resulting in a shorter target recognition sequence of 17-18 in the sgRNA molecule, and is more sensitivity to mismatched bases. It may reduce off-target mutation rates while maintaining the efficiency of on-target modifications (Fu et al., Nat Biotechnol. 2014; 32:279-284; PCT patent publication NO: 2014144592; the content of each of which is incorporated by reference in their entirety).

In some embodiments, a sgRNA used for the CRISPR-Cas system may be a synthetic tracrRNA and crRNA molecule with sequence properties described in the PCT patent publication NO.: 2015112896, the content of which is incorporated by reference in its entirety. Such synthetic tracrRNA molecule may comprising from 5′ to 3′ an optional anti-zipper sequence comprising at least about 3 nucleotides, a bulge sequence comprising at least about 3 nucleotides, an anti-stitch sequence comprising a nucleotide sequence of NNANN; a nexus sequence and a hairpin sequence comprising a nucleotide sequence having at least one hairpin. Accordingly, such synthetic crRNA molecule may comprising from 5′ to 3′ an optional sipper sequence comprising at least about 3 nucleotides that when present hybridizes to the anti-zippper sequence of the synthetic tracrRNA, a bulge sequence, a stitch sequence comprising a nucleotide sequence of NNUNN that hybridizes to the anti-stitch sequence of the synthetic tracrRNA, a nucleotide G and a spacer sequence. In some embodiments, a nucleic acid array comprising multiple copies of the synthetic tracrRNAs or crRNAs may be included in the present conjugate. In other embodiments, a chimeric nucleic acid molecule comprising the synthetic tracrRNA and the synthetic crRNA may be included in the present invention.

In some embodiments, the guide RNA may comprise two separate RNA molecules; one RNA molecule contains the tracr sequence and the other is capable of hybridize to a target motif of a selected polynucleotide sequence. The two RNA molecules can further form a duplex which is capable of directing a Cas protein to and hybridizing the target motif of the target polynucleotide sequence. In some embodiments, at least one of the RNA molecules comprises tracrRNA. In some embodiments, at least one of the RNA molecules comprises CRISPR RNA (crRNA). As a non-limiting example, one tracrRNA molecule, together with one, two, three, or multiple crRNAs may be included into one conjugate; the conjugate comprising such tracrRNA and crRNA may be served as the guide RNA of a Cas protein when applied together to a target polynucleotide sequence.

The sgRNA may be selected from numerous libraries that contain sgRNAs against the genes in a given genome. For examples, the payload may be sgRNAs from a sgRNA library that contains 73,000 sgRNAs against 7,114 genes that was used for genetic screens in human cells (Wang et al., Science, 2014; 343:80-84), or sgRNAs from a library that contains 87,897 sgRNAs targeting 19,150 mouse genes for comprehensive loss-of-function screening in mice (Koike-Yusa et al., Nat Biotechnol. 2014; 32:267-273).

Other guide RNAs that can be included in the present conjugate may include those disclosed in the US patent publication NOs.: 20150176013; the content of which is incorporated herein by reference in its entirety.

In some embodiments, a sgRNA may be designed according to any known sgRNA design softwares developed in the art, such as Target Finder from Dr. Feng Zhang, E-CRISP (Heigwer et al., Nature Methods, 2014; 11: 122-123), Cas-OFFinder (Bae et al., Bioinformatics, 2014, 30:1473-1475), CasFinder (Aach et al., BioRxiv, doi: http://dx.doi.org/10.1101/005074).

In some embodiments, the sgRNA comprises masked nucleotide derivatives called pro-nucleotides, which were converted in the living cells into biologically active nucleotides. In one non-limiting example, the masked nucleotide may comprise 3′-azido-3′-deoxythymidine (AZT), 2′,3′-dideoxyuridine (ddU), 2′,3′-dideoxyadenosine (ddA), 2′,3′-dideoxyinosine (ddI), 2′,3′-dideoxy-2′,3′-didehydrothymidine (d4T), 9-[9(1,3-dihydroxy-2-propoxy)methyl]guanine, acyclovir (ACV), 2′,3′-dideoxycytidine (ddC), 2′,3′-dideoxy-3′-thiacytidine (3TC). In another non-limiting example, the masked nucleotide may comprise any pro-nucleotide disclosed in US20130316970 to Kraszewski et al., the contents of which are incorporated herein by reference in their entirety, such as any of formulas (I)-(XVI). In yet another non-limiting example, the masked nucleotide may comprise any nucleotide mimic prodrugs disclosed in WO 2003072757 to Ariza et al., the contents of which are incorporated herein by reference in their entirety, such as lipid-masked nucleotide mimics, in which a lipid is attached to the terminal phosphorus of a nucleotide mimic directly or through a biologically-cleavable linker.

### B. Linkers

The conjugates contain one or more linkers attaching the active agents and targeting moieties. The linker, Y, is bound to one or more active agents and a targeting ligand to form a conjugate, wherein the conjugate releases at least one active agent upon delivery to a target cell. The linker Y is attached to the targeting moiety X and the active agent Z by functional groups independently selected from an ester bond, disulfide, amide, acylhydrazone, ether, carbamate, carbonate, and urea. Alternatively the linker can be attached to either the targeting moiety or the active drug by a non-cleavable group such as provided by the conjugation between a thiol and a maleimide, an azide and an alkyne. The linker is independently selected from the group consisting alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl, ether, alkoxy, aryloxy, amino, amide, carbamate, alkyl, alkenyl, alkynyl, aryl, arylalkyl, cycloalkyl, heteroaryl, or heterocyclyl is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl.

In some embodiments, the linker can be a C₁-C₁₀ straight chain alkyl, C₁-C₁₀ straight chain O-alkyl, C₁-C₁₀ straight chain substituted alkyl, C₁-C₁₀ straight chain substituted O-alkyl, C₄-C₁₃ branched chain alkyl, C₄-C₁₃ branched chain O-alkyl, C₂-C₁₂ straight chain alkenyl, C₂-C₁₂ straight chain O-alkenyl, C₃-C₁₂ straight chain substituted alkenyl, C₃-C₁₂ straight chain substituted O-alkenyl, polyethylene glycol, polylactic acid, polyglycolic acid, poly(lactide-co-glycolide), polycarprolactone, polycyanoacrylate, ketone, aryl, heterocyclic, succinic ester, amino acid, aromatic group, ether, crown ether, urea, thiourea, amide, purine, pyrimidine, bypiridine, indole derivative acting as a cross linker, chelator, aldehyde, ketone, bisamine, bis alcohol, heterocyclic ring structure, azirine, disulfide, thioether, hydrazone and combinations thereof. For example, the linker can be a C₃ straight chain alkyl or a ketone. The alkyl chain of the linker can be substituted with one or more substituents or heteroatoms. In some embodiments, the alkyl chain of the linker may optionally be interrupted by one or more atoms or groups selected from —O—, —C(═O)—, —NR, —O—C(═O)—NR—, —S—, —S—S—.

In some embodiments, the linker may be cleavable and is cleaved to release the active agent. The cleavable functionality may be hydrolyzed in vivo or may be designed to be hydrolyzed enzymatically, for example by Cathepsin B. A “cleavable” linker, as used herein, refers to any linker which can be cleaved physically or chemically. Examples for physical cleavage may be cleavage by light, radioactive emission or heat, while examples for chemical cleavage include cleavage by re-dox-reactions, hydrolysis, pH-dependent cleavage.

In one embodiment, the linker may be cleaved by an enzyme. As a non-limiting example, the linker may be a polypeptide moiety, e.g. AA in WO2010093395 to Govindan, the content of which is incorporated herein by reference in its entirety; that is cleavable by intracellular peptidase. Govindan teaches AA in the linker may be a di, tri, or tetrapeptide such as Ala-Leu, Leu-Ala-Leu, and Ala-Leu-Ala-Leu. In another example, the cleavable linker may be a branched peptide. The branched peptide linker may comprise two or more amino acid moieties that provide an enzyme cleavage site. Any branched peptide linker disclosed in WO1998019705 to Dubowchik, the content of which is incorporated herein by reference in its entirety, may be used as a linker in the conjugate of the present invention. As another example, the linker may comprise a lysosomally cleavable polypeptide disclosed in U.S. Pat. No. 8,877,901 to Govindan et al., the content of which is incorporated herein by reference in its entirety. As another example, the linker may comprise a protein peptide sequence which is selectively enzymatically cleavable by tumor associated proteases, such as any Y and Z structures disclosed in U.S. Pat. No. 6,214,345 to Firestone et al., the content of which is incorporated herein by reference in its entirety.

In one embodiment, the cleaving of the linker is non-enzymatic. Any linker disclosed in US 20110053848 to Cleemann et al., the contents of which are incorporated herein by reference in their entirety, may be used. For example, the linker may be a non-biologically active linker represented by formula (I).

In one embodiment, the linker may be a beta-glucuronide linker disclosed in US 20140031535 to Jeffrey, the contents of which are incorporated herein by reference in their entirety. In another embodiment, the linker may be a self-stabilizing linker such as a succinimide ring, a maleimide ring, a hydrolyzed succinimide ring or a hydrolyzed maleimide ring, disclosed in US20130309256 to Lyon et al., the contents of which are incorporated herein by reference in their entirety. In another embodiment, the linker may be a human serum albumin (HAS) linker disclosed in US 20120003221 to McDonagh et al., the contents of which are incorporated herein by reference in their entirety. In another embodiment, the linker may comprise a fullerene, e.g., C₆₀, as disclosed in US 20040241173 to Wilson et al., the contents of which are incorporated herein by reference in their entirety. In another embodiment, the linker may be a recombinant albumin fused with polycysteine peptide as disclosed in U.S. Pat. No. 8,541,378 to Ahn et al., the contents of which are incorporated herein by reference in their entirety. In another embodiment, the linker comprises a heterocycle ring. For example, the linker may be any heterocyclic 1,3-substituted five- or six-member ring, such as thiazolidine, disclosed in US 20130309257 to Giulio, the content of which is incorporated herein by reference in its entirety.

In some embodiments, the linker Y may be a Linker Unit (LU) as described in US2011/0070248, the contents of which are incorporated herein by reference in their entirety. In formula (I) where the Ligand Drug Conjugate has formula L-(LU-D)ₚ the targeting moiety X corresponds to L (the Ligand unit) and the active agent Z (e.g., sgRNAs) corresponds to D (the drug unit).

In some embodiments, the linker Y may be Aₘ and the conjugate can be a compound according to Formula Ia:

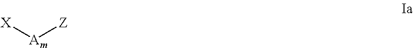

wherein A is defined herein, m=0-20.

A in Formula Ia is a spacer unit, either absent or independently selected from the following substituents. For each substituent, the dashed lines represent substitution sites with X, Z or another independently selected unit of A wherein the X, Z, or A can be attached on either side of the substituent:

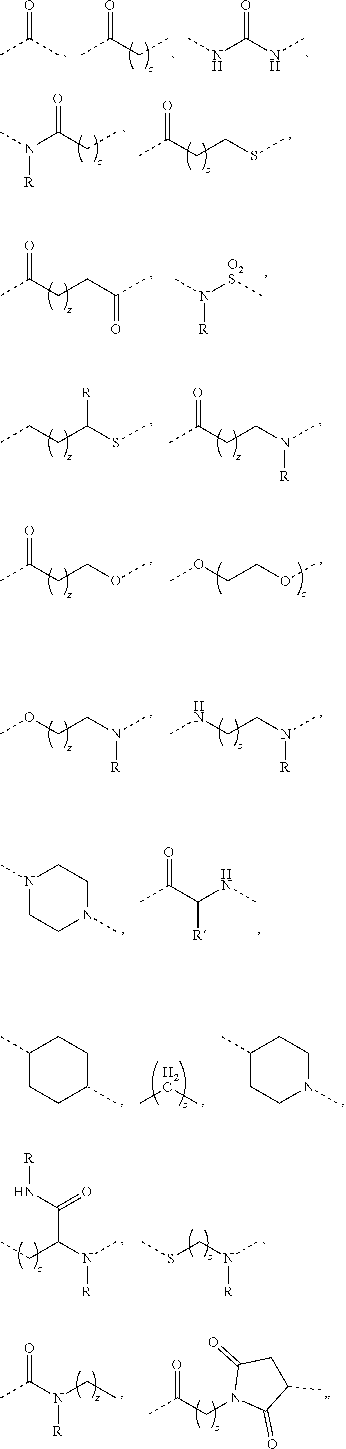

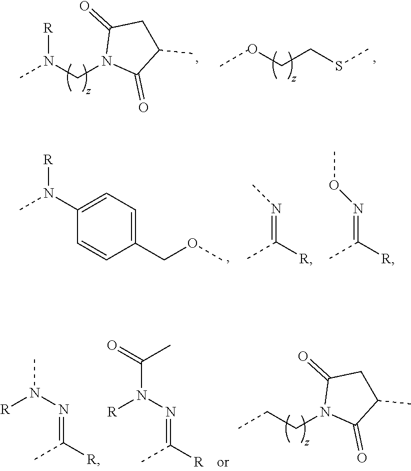

wherein z=0-40, R is H or an optionally substituted alkyl group, and R′ is any side chain found in either natural or unnatural amino acids.

The linker may be selected from dicarboxylate derivatives of succinic acid, glutaric acid or diglycolic acid. In some embodiments, the linker Y may be X′—R¹—Y′—R²—Z′ and the conjugate can be a compound according to Formula Ib:

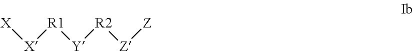

wherein X is a targeting moiety defined herein below; Z is an active agent (e.g., guide RNA); X′, R¹, Y′, R² and Z′ are as defined herein.

X′ is either absent or independently selected from carbonyl, amide, urea, amino, ester, aryl, arylcarbonyl, aryloxy, arylamino, one or more natural or unnatural amino acids, thio or succinimido; R¹ and R² are either absent or comprised of alkyl, substituted alkyl, aryl, substituted aryl, polyethylene glycol (2-30 units); Y′ is absent, substituted or unsubstituted 1,2-diaminoethane, polyethylene glycol (2-30 units) or an amide; Z′ is either absent or independently selected from carbonyl, amide, urea, amino, ester, aryl, arylcarbonyl, aryloxy, arylamino, thio or succinimido. In some embodiments, the linker can allow one active agent molecule to be linked to two or more ligands, or one ligand to be linked to two or more active agent molecule.

In some embodiments, the linker may be used with compositions of the invention are well known in the art, and include, e.g., thyroglobulin, albumins such as human serum albumin, tetanus toxoid, polyamino acid residues such as poly L-lysine, poly L-glutamic acid, influenza virus proteins, hepatitis B virus core protein, and the like.

In some embodiments, the linker may be a hydrophilic linker as disclosed by Zhao et al. in PCT patent publication NO., WO2014/080251; the content of which is incorporated by reference in its entirety. The hydrophilic linkers may contain phosphinate, sulfonyl, and/or sulfoxide groups to link active agents (payloads) to a cell-targeting moiety.

In other embodiments, the linker promotes cellular internalization. In certain embodiments, the linker promotes cellular internalization. A variety of linkers that can be used with the present compositions and methods are described in WO 2004/010957, US2012/0141509, and US2012/0288512, which are incorporated by reference herein in their entirety.

The conjugate X—Y—Z can be a conjugate as described in WO2014/134486, the contents of which are incorporated herein by reference in their entirety. The targeting moiety X, corresponds to the cell binding agent, CBA in formula (I′) or (I) as reproduced here, wherein the linker Y and the sgRNA molecules Z together correspond to the remainder of the formula (in parentheses).

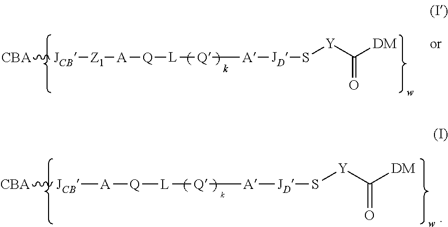

The conjugate X—Y—Z can be a conjugate as described in U.S. Pat. No. 7,601,332, the contents of which are incorporated herein by reference in their entirety, wherein conjugates are described as follows, and the targeting moiety X corresponds to V (the vitamin receptor binding moiety) and the linker Y corresponds to the bivalent linker (L) which can comprise one or more components selected from spacer linkers (ls), releasable linkers (lr), and heteroatom linkers (lH), and combinations thereof, in any order:

### V-L-D

V-(lᵣ)_{c}-D
V-(lₛ)ₐ-D
V-(lₛ)ₐ-(lᵣ)_{c}-D
V-(lᵣ)_{c}-(lₛ)ₐ-D
V-(l_{H})_{b}-(lᵣ)_{c}-D
V-(lᵣ)_{c}-(l_{H})_{b}-D
V-(l_{H})_{d}-(lᵣ)_{c}-(l_{H})ₑ-D
V-(lₛ)ₐ-(l_{H})_{b}-(lᵣ)_{c}-D
V-(lᵣ)_{c}-(l_{H})_{b}-(lₛ)ₐ-D
V-(l_{H})_{d}-(lₛ)ₐ-(lᵣ)_{c}-(l_{H})ₑ-D
V-(l_{H})_{d}-(lᵣ)_{c}-(lₛ)ₐ-(l_{H})ₑ-D
V-(l_{H})_{d}-(lₛ)ₐ-(l_{H})_{b}-(l_{H})ₑ-(l_{H})ₑ-D
V-(l_{H})_{d}-(lᵣ)_{c}-(l_{H})_{b}-(lₛ)ₐ-(l_{H})ₑ-D
V-(lₛ)ₐ-(lᵣ)_{c}-(l_{H})_{b}-D
V-[(lₛ)ₐ-(l_{H})_{b}]_{d}-(lᵣ)_{c}-(l_{H})ₑ-D

In some embodiments, the linker may comprise a complexing agent for sgRNA disclosed in U.S. Pat. No. 8,772,471 to Shankar et al., the contents of which are incorporated herein by reference in their entirety, including poly-amino acids, polyimines, polyacrylates, polyalkylacrylates, polyoxethanes, polyalkylcyanoacrylates, cationized gelatins, albumins, starches, acrylates, polyethyleneglycols (PEG) and starches, polyalkylcyanoacrylates, DEAE-derivatized polyimines, pollulans, celluloses and starches, chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DE AE-albumin and DEAE-dextran, polymethylacrylate, polyhexylacrylate, poly(D,L-lactic acid), poly(DL-lactic-co-glycolic acid (PLGA), alginate, and polyethyleneglycol (PEG), and polyethylenimine.

In some embodiments, the linker may comprise a pharmacokinetic modulator consisting of a hydrophobic group having 16 or more carbon atoms, e.g. 16 to 20 carbon atoms, as disclosed in US 2012/0157509 to Hadwiger et al., the contents of which are incorporated herein by reference in their entirety. For example, the pharmacokinetic modulator may be selected from the group consisting of: palmitoyl, hexadec-8-enoyl, oleyl, (9E,12E)-octadeca-9,12-dienoyl, dioctanoyl, C16-C20 acyl, and cholesterol. The linker may further comprise a lysine or ornithine between the pharmacokinetic modulator and the targeting moiety.

In some embodiments, the linker may comprise a cell-penetrating peptide, also called cell-permeable peptide, protein-transduction domain (PTD) or membrane-translocation sequences (MTS), to facilitate the cellular uptake of the conjugates of the invention. Cell-penetrating peptides are peptides that are capable of crossing biological membrane or a physiological barrier. They can direct conjugates of the present invention to a desired cellular destination, e.g. into the nucleus. Cell-penetrating peptides can be any suitable length, such as less than or equal to about 500, 250, 150, 100, 50, 25, 10 or 5 amino acids in length. For example, they may be 4, 5, 6, 7, 5, 6, 7, 8, 9, 10, 11, 12, 13, 14, 15, 16, 17, 18, 19, 20, 21, 22, 23, 24 or 25 amino acids in length. They may be cationic or amphiphilic and may be arginine or lysine rich.

In some embodiments, the linker of the conjugate may be optional. In this context, the active agent and the targeting moiety of the conjugate are directly connected to each other.

### C. Targeting Moieties

In accordance with the present invention, a conjugate can contain one or more targeting moieties or targeting ligands. For example, the conjugate can include an active agent with multiple targeting moieties each attached via a different linker. The conjugate can have the structure X—Y—Z—Y—X where each X is a targeting moiety that may be the same or different, each Y is a linker that may be the same or different, and Z is the active agent (payload). In some embodiments, the targeting moiety does not substantially interfere with efficacy of the active agent Z in vivo. In some aspects, the targeting moiety may contain adjuvant activity, in addition to targeted binding to a cell of interest.

Targeting ligands or moieties can be polypeptides (e.g., antibodies), peptides, antibody mimetics, nucleic acids (e.g., aptamers), glycoproteins, small molecules, carbohydrates, lipids, nanoparticles.

In some embodiments, the targeting moiety, X, may be other peptides such as somatostatin, octeotide, LHRH (luteinizing hormone releasing hormone), epidermal growth factor receptor (EGFR) binding peptide, aptide or bipodal peptide, RGD-containing peptides, a protein scaffold such as a fibronectin domain, a single domain antibody, a stable scFv, or other homing peptides. As non-limiting examples, a protein or peptide based targeting moiety may be a protein such as thrombospondin, tumor necrosis factors (TNF), annexin V, an interferon, angiostatin, endostatin, cytokine, transferrin, GM-CSF (granulocyte-macrophage colony-stimulating factor), or growth factors such as vascular endothelial growth factor (VEGF), hepatocyte growth factor (HGF), (platelet-derived growth factor (PDGF), basic fibroblast growth factor (bFGF), and epidermal growth factor (EGF).

In some embodiments, the targeting moiety is an antibody, an antibody fragment, RGD peptide, folic acid or prostate specific membrane antigen (PSMA). In some embodiments, the protein scaffold may be an antibody-derived protein scaffold. Non-limiting examples include single domain antibody (dAbs), nanobody, single-chain variable fragment (scFv), antigen-binding fragment (Fab), Avibody, minibody, CH2D domain, Fcab, and bispecific T-cell engager (BiTE) molecules. In some embodiments, scFv is a stable scFv, wherein the scFv has hyperstable properties. In some embodiments, the nanobody may be derived from the single variable domain (VHH) of camelidae antibody.

In some embodiments, the protein scaffold may be a non-antibody-derived protein scaffold, wherein the protein scaffold is based on nonantibody binding proteins. The protein scaffold may be based on engineered Kunitz domains of human serine protease inhibitors (e.g., LAC1-D1), DARPins (designed ankyrin repeat domains), avimers created from multimerized low-density lipoprotein receptor class A (LDLR-A), anticalins derived from lipocalins, knottins constructed from cysteine-rich knottin peptides, affibodies that are based on the Z-domain of staphylococcal protein A, adnectins or monobodies and pronectins based on the 10^{th} or 14^{th} extracellular domain of human fibronectin III, Fynomers derived from SH3 domains of human Fyn tyrosine kinase, or nanofitins (formerly Affitins) derived from the DNA binding protein Sac7d.

In some embodiments, the protein scaffold may be based on a fibronectin domain. In some embodiments, the protein scaffold may be based on fibronectin type III (FN3) repeat protein. In some embodiments, the protein scaffold may be based on a consensus sequence of multiple FN3 domains from human Tenascin-C(hereinafter “Tenascin”). Any protein scaffold based on a fibronectin domain disclosed in U.S. Pat. No. 8,569,227 to Jacobs et al., the content of which is incorporated herein by reference in its entirety; may be used as a targeting moiety of the conjugate of the invention.

In some embodiments, the protein scaffold may be any protein scaffold disclosed in Mintz and Crea, BioProcess, vol. 11(2):40-48 (2013), the contents of which are incorporated herein by reference in their entirety. Any of the protein scaffolds disclosed in Tables 2-4 of Mintz and Crea may be used as a targeting moiety of the conjugate of the invention.

In some embodiments, the targeting moiety is an arginylglycylaspartic acid (RGD) peptide, a tripeptide composed of L-arginine, glucine and L-aspartic acid, which is a common cell targeting element for cellular attachment via integrins.

In some embodiments, the targeting moiety or targeting ligand may be any moledule that can bind to luteinizing-hormone-releasing hormone receptor (LHRHR). Such targeting ligands can be peptides, antibody mimetics, nucleic acids (e.g., aptamers), polypeptides (e.g., antibodies), glycoproteins, small molecules, carbohydrates, or lipids. In some embodiments, the targeting moiety is LHRH or a LHRH analog.

Luteinizing-hormone-releasing hormone (LHRH), also known as gonadotropin-releasing hormone (GnRH) controls the pituitary release of gonadotropins (LH and FSH) that stimulate the synthesis of sex steroids in the gonads. LHRH is a 10-amino acid peptide that belongs to the gonadotropin-releasing hormone class. Signaling by LHRH is involved in the first step of the hypothalamic-pituitary-gonadal axis. An approach in the treatment of hormone-sensitive tumors directed to the use of agonists and antagonists of LHRH (A. V. Schally and A. M. Comaru-Schally. Sem. Endocrinol., 5 389-398, 1987) has been reported. Some LHRH agonists, when substituted in position 6, 10, or both are much more active than LHRH and also possess prolonged activity. Some LHRH agonists are approved for clinical use, e.g., Leuprolide, triptorelin, nafarelin and goserelin.

Some human tumors are hormone dependent or hormone-responsive and contain hormone receptors. Certain of these tumors are dependent on or responsive to sex hormones or growth factors, or have components that are dependent or responsive to such hormones. Mammary carcinomas contain estrogen, progesterone, glucocorticoid, LHRH, EGF IGF-I and somatostatin receptors. Peptide hormone receptors have been detected in acute leukaemia, prostate-, breast-, pancreatic, ovarian-, endometri cancer, colon cancer and brain tumors (M. N. Pollak, et al., Cancer Lett. 38 223-230 1987; F. Pekonen, et al., Cancer Res., 48 1343-1347, 1988; M. Fekete, et al., J Clin. Lab. Anal. 3 137-147, 1989; G. Emons, et al., Eur. J. Cancer Oncol., 25215-221 1989). It has been found (M. Fekete, et al., Endocrinology. 124 946-955. 1989; M Fekete, et al. Pancreas 4521-528, 1989) that both agonistic and antagonistic analog of LHRH bind to human breast cancer cell membranes, and also to the cell membranes of pancreatic cancer. It has been demonstrated that biologically active peptides such a melanotropin (MSH), epidermal growth factor, insulin and agonistic and antagonisti analogs of LHRH (L Jennes, et. al., Peptides 5 215-220, 1984) are internalized b their target cells by endocytosis.

The conjugates of the invention can employ any of the large number of known molecules that recognize the LHRH receptor, such as known LHRH receptor agonists and antagonists. In some embodiments, the LHRH analog portion of the conjugate contains between 8 and 18 amino acids.

Examples of LHRH binding molecules useful in the present invention are described herein. Further non-limiting examples are analogs of pyroGlu-His-Trp-Ser-Tyr-Gly-Leu-Arg-Pro-Gly-NH2, leuprolide, triptorelin, nafarelin, buserelin, goserelin, cetrorelix, ganirelix, azaline-B, degarelix and abarelix.

Methods for synthesizing LHRH peptides and analogs are well documented and are within the ability of a person of ordinary skill in the art as exemplified in the references listed supra. Further synthetic procedures are provided in the following examples. The following examples also illustrate methods for synthesizing the targeted cytotoxic compounds of the present invention. Specific targeting of therapeutic or cytotoxic agents allows selective destruction of a tumor expressing a receptor specific for a biologically active peptide. For example, a tumor expressing a LHRH receptor includes a neoplasm of the lung, breast, prostate, colon, brain, gastrointestinal tract, neuroendocrine axis, liver, or kidney (see Schaer et al., Int. J. Cancer, 70:530-537, 1997; Chave et al., Br. J. Cancer 82(1):124-130, 2000; Evans et al., Br. J. Cancer 75(6):798-803, 1997).

In some embodiments, the targeting moiety, e.g., LHRH analog, used in the invention is hydrophilic, and is therefore water soluble. In some embodiments, such conjugates and particles containing such conjugates are used in treatment paradigms in which this feature is useful, e.g., compared to conjugates comprising hydrophobic analogs. Hydrophilic analogs described herein can be soluble in blood, cerebrospinal fluid, and other bodily fluids, as well as in urine, which may facilitate excretion by the kidneys. This feature can be useful, e.g., in the case of a composition that would otherwise exhibit undesirable liver toxicity. The invention also discloses specific hydrophilic elements (e.g., incorporation of a PEG linker, and other examples in the art) for incorporation into peptide analogs, allowing modulation of the analog's hydrophilicity to adjust for the chemical and structural nature of the various conjugated cytotoxic agents.

In some embodiments, the targeting moiety is an antibody mimetic such as a monobody, e.g., an ADNECTIN™ (Bristol-Myers Squibb, New York, N.Y.), an Affibody® (Affibody AB, Stockholm, Sweden), Affilin, nanofitin (affitin, such as those described in WO 2012/085861, an Anticalin™, an avimers (avidity multimers), a DARPin™ a Fynomer™, Centyrin™, and a Kunitz domain peptide. In certain cases, such mimetics are artificial peptides or proteins with a molar mass of about 3 to 20 kDa. Nucleic acids and small molecules may be antibody mimetic.

In another example, a targeting moiety can be an aptamer, which is generally an oligonucleotide (e.g., DNA, RNA, or an analog or derivative thereof) that binds to a particular target, such as a polypeptide. In some embodiments, the targeting moiety is a polypeptide (e.g., an antibody that can specifically bind a tumor marker). In certain embodiments, the targeting moiety is an antibody or a fragment thereof. In certain embodiments, the targeting moiety is an Fc fragment of an antibody.

In another example, a targeting moiety may be a non-immunoreactive ligand. For example, the non-immunoreactive ligand may be insulin, insulin-like growth factors I and II, lectins, apoprotein from low density lipoprotein, etc. as disclosed in US 20140031535 to Jeffrey, the contents of which are incorporated herein by reference in their entirety. Any protein or peptide comprising a lectin disclosed in WO2013181454 to Radin, the contents of which are incorporated herein by reference in their entirety, may be used as a targeting moiety.

In another example, the conjugate of the invention may target a hepatocyte intracellularly and a hepatic ligand may be used as a targeting moiety. Any hepatic ligand disclosed in US 20030119724 to Ts'o et al., the contents of which are incorporated herein by reference in their entirety, such as the ligands in FIG. 1, may be used. The hepatic ligand specifically binds to a hepatic receptor, thereby directing the conjugate into cells having the hepatic receptor.

In another example, a targeting moiety may interact with a protein that is overexpressed in tumor cells compared to normal cells. The targeting moiety may bind to a chaperonin protein, such as Hsp90, as disclosed in US 20140079636 to Chimmanamada et al., the contents of which are incorporated herein by reference in their entirety. The targeting moiety may be an Hsp90 inhibitor, such as geldanamycins, macbecins, tripterins, tanespimycins, and radicicols.

In another example, the conjugate may have a terminal half-life of longer than about 72 hours and a targeting moiety may be selected from Table 1 or 2 of US 20130165389 to Schellenberger et al., the contents of which are incorporated herein by reference in their entirety. The targeting moiety may be an antibody targeting delta-like protein 3 (DLL3) in disease tissues such as lung cancer, pancreatic cancer, skin cancer, etc., as disclosed in WO2014125273 to Hudson, the contents of which are incorporated herein by reference in their entirety. The targeting moiety may also any targeting moiety in WO2007137170 to Smith, the contents of which are incorporated herein by reference in their entirety. The targeting moiety binds to glypican-3 (GPC-3) and directs the conjugate to cells expressing GPC-3, such as hepatocellular carcinoma cells.

In some embodiments, a target of the targeting moiety may be a marker that is exclusively or primarily associated with a target cell, or one or more tissue types, with one or more cell types, with one or more diseases, and/or with one or more developmental stages. In some embodiments, a target can comprise a protein (e.g., a cell surface receptor, transmembrane protein, glycoprotein, etc.), a carbohydrate (e.g., a glycan moiety, glycocalyx, etc.), a lipid (e.g., steroid, phospholipid, etc.), and/or a nucleic acid (e.g., a DNA, RNA, etc.).

In another embodiment, targeting moieties may be peptides for regulating cellular activity. For example, the targeting moiety may bind to Toll Like Receptor (TLR). It may be a peptide derived from vaccinia virus A52R protein such as a peptide comprising SEQ ID No. 13 as disclosed in U.S. Pat. No. 7,557,086, a peptide comprising SEQ ID No. 7 as disclosed in U.S. Pat. No. 8,071,553 to Hefeneider, et al., or any TLR binding peptide disclosed in WO 2010141845 to McCoy, et al., the contents of each of which are incorporated herein by reference in their entirety. The A52R derived synthetic peptide may significantly inhibit cytokine production in response to both bacterial and viral pathogen associated molecular patterns, and may have application in the treatment of inflammatory conditions that result from ongoing toll-like receptor activation,

In another embodiment, targeting moieties many be amino acid sequences or single domain antibody fragments for the treatment of cancers and/or tumors. For example, targeting moieties may be an amino acid sequence that binds to Epidermal Growth Factor Receptor 2 (HER2). Targeting moieties may be any HER2-binding amino acid sequence described in US 20110059090, U.S. Pat. No. 8,217,140, and U.S. Pat. No. 8,975,382 to Revets, et al., the contents of each of which are incorporated herein by reference in their entirety. The targeting moiety may be a domain antibody, a single domain antibody, a VHH, a humanized VHH or a camelized VH.

In another embodiment, targeting moieties may be peptidomimetic macrocycles for the treatment of disease. For example, targeting moieties may be peptidomimetic macrocycles that bind to the growth hormone-releasing hormone (GHRH) receptor, such as a peptidomimetic macrocycle comprising an amino acid sequence which is at least about 60% identical to GHRH 1-29 and at least two macrocycle-forming linkers as described in US20130123169 to Kawahata et al., the contents of which are incorporated herein by reference in their entirety. In another embodiment, the peptidomimetic macrocycle targeting moiety may be prepared by introducing a cross-linker between two amino acid residues of a polypeptide as described in US 20120149648 and US 20130072439 to Nash et al., the contents of each of which are incorporated herein by reference in their entirety. Nash et al. teaches that the peptidomimetic macrocycle may comprise a peptide sequence that is derived from the BCL-2 family of proteins such as a BH3 domain. The peptidomimetic macrocycle may comprise a BID, BAD, BIM, BIK, NOXA, PUMA peptides.

In another embodiment, targeting moieties may be polypeptide analogues for transport to cells. For example, the polypeptide may be an Angiopep-2 polypeptide analog. It may comprising a polypeptide comprising an amino acid sequence at least 80% identical to SEQ ID No.97 as described in US 20120122798 to Castaigne et al., the contents of which are incorporated herein by reference in their entirety. Additionally, polypeptides may transport to cells, such as liver, lung, kidney, spleen, and muscle, such as Angiopep-4b, Angiopep-5, Angiopep-6, and Angiopep-7 polypeptide as described in EP 2789628 to Beliveau et al., the contents of each of which are incorporated herein by reference in their entirety.

In another embodiment, targeting moieties may be homing peptides to target liver cells in vivo. For example, the melittin delivery peptides that are administered with RNAi polynucleotides as described in U.S. Pat. No. 8,501,930 Rozema, et al., the contents of which are incorporated herein by reference in their entirety, may be used as targeting moieties. In addition, delivery polymers provide membrane penetration function for movement of the RNAi polynucleotides from the outside the cell to inside the cell as described in U.S. Pat. No. 8,313,772 to Rozema et al., the contents of each of which are incorporated herein by reference in their entirety. Any delivery peptide disclosed by Rozema et al. may be used as targeting moeities.

In another embodiment, targeting moieties may be structured polypeptides to target and bind proteins. For example, polypeptides with sarcosine polymer linkers that increase the solubility of structured polypeptides, as described in WO 2013050617 to Tite, et al., the contents of which are incorporated herein by reference in their entirety, may be used as targeting moieties. Additionally, polypeptide with variable binding activity produced by the methods described in WO 2014140342 to Stace, et al., the contents of which are incorporated herein by reference in their entirety. The polypeptides may be evaluated for the desired binding activity.

In another embodiment, modifications of the targeting moieties affect a compound's ability to distribute into tissues. For example, a structure activity relationship analysis was completed on a low orally bioavailable cyclic peptide and the permeability and clearance was determined as described in Rand, A C., et al., Medchemcomm. 2012, 3(10): 1282-1289, the contents of which are incorporated herein by reference in their entirety. Any of the cyclic peptide disclosed by Rand et al., such as N-methylated cyclic hexapeptides, may be used as targeting moieties.

In another embodiment, targeting moieties may be a polypeptide which is capable of internalization into a cell. For example, targeting moieties may be an Alphabody capable of internalization into a cell and specifically binding to an intracellular target molecule as described in US 20140363434 to Lasters, et al., the contents of which are incorporated herein by reference in their entirety. As taught by Lasters et al., an ‘Alphabody’ or an ‘Alphabody structure’ is a self-folded, single-chain, triple-stranded, predominantly alpha-helical, coiled coil amino acid sequence, polypeptide or protein. The Alphabody may be a parallel Alphabody or an anti-parallel Alphabody. Moreover, targeting moieties may be any Alphabody in the single-chain Alphabody library used for the screening for and/or selection of one or more Alphabodies that specifically bind to a target molecule of interest as described in WO 2012092970 to Desmet et al., the contents of which are incorporated herein by reference in their entirety.

In another embodiment, targeting moieties may consist of an affinity-matured heavy chain-only antibody. For example, targeting moieties may be any VII heavy chain-only antibodies produced in a transgenic non-human mammal as described in US 20090307787 to Grosveld et al., the contents of which are incorporated herein by reference in their entirety.

In another embodiment, targeting moieties may bind to the hepatocyte growth factor receptor “HGFr” or “cMet”. For example, targeting moieties may be a polypeptide moiety that is conjugated to a detectable label for diagnostic detection of cMet as described in U.S. Pat. No. 9,000,124 to Dransfield et al., the contents of which are incorporated herein by reference in their entirety. Additionally, targeting moieties may bind to human plasma kallikrein and may comprise BPTI-homologous Kunitz domains, especially LACI homologues, to bind to one or more plasma (and/or tissue) kallikreins as described in WO 1995021601 to Markland et al., the contents of which are incorporated herein by reference in their entirety.

In another embodiment, targeting moieties are evolved from weak binders and anchor-scaffold conjugates having improved target binding and other desired pharmaceutical properties through control of both synthetic input and selection criteria. Any target binding element identified in US 20090163371 to Stern et al., the contents of which are incorporated herein by reference in their entirety, may be used as a targeting moiety. Moreover, targeting moieties may be macrocyclic compounds that bind to inhibitors of apoptosis as described in WO 2014074665 to Borzilleri et al., the contents of which are incorporated herein by reference in their entirety.

In another embodiment, targeting moieties may comprise pre-peptides that encode a chimeric or mutant lantibiotic. For example, targeting moieties may be pre-tide that encode a chimera that was accurately and efficiently converted to the mature lantibiotic, as demonstrated by a variety of physical and biological activity assays as described in U.S. Pat. No. 5,861,275 to Hansen, the contents of which are incorporated herein by reference in their entirety. The mixture did contain an active minor component with a biological activity.

In some embodiments, the protein scaffold may be any protein scaffold disclosed in Mintz and Crea, BioProcess, vol. 11(2):40-48 (2013), the contents of which are incorporated herein by reference in their entirety. Any of the protein scaffolds disclosed in Tables 2-4 of Mintz and Crea may be used as a targeting moiety of the conjugate of the invention.

In another embodiment, targeting moieties may comprise a leader peptide of a recombinant manganese superoxide dismutase (rMnSOD-Lp). For example, rMnSOD-Lp which delivers cisplatin directly into tumor cells as described in Borrelli, A., et al., Chem Biol Drug Des. 2012, 80(1):9-16, the contents of which are incorporated herein by reference in their entirety, may be used a targeting moiety.

In another embodiment, the targeting moiety may be an antibody for the treatment of glioma. For example, an antibody or antigen binding fragment which specifically binds to JAMM-B or JAM-C as described in U.S. Pat. No. 8,007,797 to Dietrich et al., the contents of which are incorporated herein by reference in their entirety, may be used as a targeting moiety. JAMs are a family of proteins belonging to a class of adhesion molecules generally localized at sites of cell-cell contacts in tight junctions, the specialized cellular structures that keep cell polarity and serve as barriers to prevent the diffusion of molecules across intercellular spaces and along the basolateral-apical regions of the plasma membrane.

In another embodiment, the targeting moiety may be a target interacting modulator. For example, nucleic acid molecules capable of interacting with proteins associated with the Human Hepatitis C virus or corresponding peptides or mimetics capable of interfering with the interaction of the native protein with the HIV accessory protein as described in WO 2011015379 and U.S. Pat. No. 8,685,652, the contents of each of which are incorporated herein by reference in their entirety, may be used as a targeting moiety.

In another embodiment, the targeting moiety may bind with biomolecules. For example, any cystine-knot family small molecule polycyclic molecular scaffolds were designed as peptidomimetics of FSH and used as peptide-vaccine as described in U.S. Pat. No. 7,863,239 to Timmerman, the contents the contents of which are incorporated herein by reference in their entirety, may be used as targeting moieties.

In another embodiment, the targeting moiety may bind to integrin and thereby block or inhibit integrin binding. For example, any highly selective disulfide-rich dimer molecules which inhibit binding of α4β7 to the mucosal address in cell adhesion molecule (MAdCAM) as described in WO 2014059213 to Bhandari, the contents of which are incorporated herein by reference in their entirety, may be used as a targeting moiety. Any inhibitor of specific integrins-ligand interactions may be used as a targeting moiety. The conjugates comprising such target moieties may be effective as anti-inflammatory agents for the treatment of various autoimmune diseases.

In another embodiment, the targeting moiety may comprise novel peptides. For example, any cyclic peptide or mimetic that is a serine protease inhibitor as described in WO 2013172954 to Wang et al., the contents of which are incorporated herein by reference in their entirety, may be used as a targeting moiety. Additionally, targeting moieties may comprise a targeting peptide that is used in the reduction of cell proliferation and the treatment of cancer. For example, a peptide composition inhibiting the trpv6 calcium channel as described in US 20120316119 to Stewart, the contents of which are incorporated herein by reference in their entirety, may be used as a targeting moiety.

In another embodiment, the targeting moiety may comprise a cyclic peptide. For example, any cyclic peptides exhibit various types of action in vivo, as described in US20100168380 and WO 2008117833 to Suga et al., and WO 2012074129 to Higuchi et al., the contents of each of which are incorporated herein by reference, may be used as targeting moieties. Such cyclic peptide targeting moieties have a stabilized secondary structure and may inhibit biological molecule interactions, increase cell membrane permeability and the peptide's half-life in blood serum.

In another embodiment, the targeting moiety may consist of a therapeutic peptide. For example, peptide targeting moieties may be an AP-1 signaling inhibitor, such as a peptide analog comprising SEQ ID No. 104 of U.S. Pat. No. 8,946,381B2 to Fear that is used for the treatment of wounds, a peptide comprising SEQ ID No. 108 in U.S. Pat. No. 8,822,409B2 to Milech, et al. that is used to treat acute respiratory distress syndrome (ARDS), or a neuroprotective AP-1 signaling inhibitory peptide that is a fusion peptide comprising a protein transduction domain having the amino acid sequence of SEQ ID NO: 1 and a peptide having the sequence of SEQ ID NO:54 as described in U.S. Pat. No. 8,063,012 to Watt, the contents of each of which are incorporated herein by reference in their entirety. In another example, the targeting moiety may be any biological modulator isolated from biodiverse gene fragment libraries as described in U.S. Pat. No. 7,803,765 and EP1754052 to Watt, any inhibitor of c-Jun dimerization as described in EP1601766 and EP1793841 to Watt, any peptide inhibitors of CD40L signaling as described in U.S. Pat. No. 8,802,634 and US20130266605 to Watt, or any peptide modulators of cellular phenotype as described in US20110218118 to Watt, the contents of each of which are incorporated herein by reference in their entirety.

In another embodiment, the targeting moiety may consist of a characterized peptide. For example, any member of the screening libraries created from bioinformatic source data to theoretically predict the secondary structure of a peptide as described in EP1987178 to Watt et al., any peptide identified from peptide libraries that are screened for antagonism or inhibition of other biological interactions by a reverse hybrid screening method as described by EP1268842 to Hopkins, et al., the contents of each of which are incorporated herein by reference in their entirety, may be used as a targeting moiety. Additionally, targeting moieties may be cell-penetrating peptides. For example, any cell-penetrating peptides linked to a cargo that are capable of passing through the blood brain barrier as described by US20140141452A1 to Watt, et al., the contents of which are incorporated herein by reference, may be used a targeting moiety.

In another embodiment, the targeting moiety may comprise a LHRH antagonist, agonist, or analog. For example, the targeting moiety may be Cetrorelix, a decapeptide with a terminal acid amide group (AC-D-Nal(2)-D-pCl-Phe-D-Pal(3)-Ser-Tyr-D-Cit-Leu-Arg-Pro-D-Ala-NH2) as described in U.S. Pat. No. 4,800,191, U.S. Pat. No. 6,716,817, U.S. Pat. No. 6,828,415, U.S. Pat. No. 6,867,191, U.S. Pat. No. 7,605,121, U.S. Pat. No. 7,718,599, U.S. Pat. No. 7,696,149 (Zentaris Ag), or pharmaceutically active decapeptides such as SB-030, SB-075 (cetrorelix) and SB-088 disclosed in EP 0 299 402 (Asta Pharma), the contents of each of which are incorporated herein by reference in their entirety. In another example, the targeting moiety may be LHRH analogues such as D-/L-MeI (4-[bis(2-chloroethyl)amino]-D/L-phenylalanine), cyclopropanealkanoyl, aziridine-2-carbonyl, epoxyalkyl, 1,4-naphthoquinone-5-oxycarbonyl-ethyl, doxorubicinyl (Doxorubicin, DOX), mitomicinyl (Mitomycin C), esperamycinyl or methotrexoyl, as disclosed in U.S. Pat. No. 6,214,969 to Janaky et al., the contents of which are incorporated herein by reference in their entirety.

In another embodiment, the targeting moiety may be any cell-binding molecule disclosed in U.S. Pat. No. 7,741,277 or U.S. Pat. No. 7,741,277 to Guenther et al. (Aeterna Zentaris), the contents of which are incorporated herein by reference in their entirety, such as octamer peptide, nonamer peptide, decamer peptide, luteinizing hormone releasing hormone (LHRH), [D-Lys6]-LHRH, LHRH analogue, LHRH agonist, Triptorelin ([D-Trp6]-LHRH), LHRH antagonist, bombesin, bombesin analogue, bombesin antagonist, somatostatin, somatostatin analogue, serum albumin, human serum albumin (HSA). These cell-binding molecules may be conjugated with disorazoles.

In another embodiment, targeting moieties may bind to growth hormone secretagogue (GHS) receptors, including ghrelin analogue ligands of GHS receptors. For example, targeting moieties may be any triazole derivatives with improved receptor activity and bioavailability properties as ghrelin analogue ligands of growth hormone secretagogue receptors as describe by U.S. Pat. No. 8,546,435 to Aicher, at al. (Aeterna Zentaris), the contents of which are incorporated herein by reference in their entirety.

In some embodiments, the targeting moiety X is an aptide or bipodal peptide. X may be any D-Aptamer-Like Peptide (D-Aptide) or retro-inverso Aptide which specifically binds to a target comprising: (a) a structure stabilizing region comprising parallel, antiparallel or parallel and antiparallel D-amino acid strands with interstrand noncovalent bonds; and (b) a target binding region I and a target binding region II comprising randomly selected n and m D-amino acids, respectively, and coupled to both ends of the structure stabilizing region, as disclosed in US Pat. Application No. 20140296479 to Jon et al., the contents of which are incorporated herein by reference in their entirety. X may be any bipodal peptide binder (BPB) comprising a structure stabilizing region of parallel or antiparallel amino acid strands or a combination of these strands to induce interstrand non-covalent bonds, and target binding regions I and II, each binding to each of both termini of the structure stabilizing region, as disclosed in US Pat. Application No. 20120321697 to Jon et al., the contents of which are incorporated herein by reference in their entirety. X may be an intracellular targeting bipodal-peptide binder specifically binding to an intracellular target molecule, comprising: (a) a structure-stabilizing region comprising a parallel amino acid strand, an antiparallel amino acid strand or parallel and antiparallel amino acid strands to induce interstrand non-covalent bonds; (b) target binding regions I and II each binding to each of both termini of the structure-stabilizing region, wherein the number of amino acid residues of the target binding region I is n and the number of amino acid residues of the target binding region II is m; and (c) a cell-penetrating peptide (CPP) linked to the structure-stabilizing region, the target binding region I or the target binding region II, as disclosed in US Pat. Application No. 20120309934 to Jon et al., the contents of which are incorporated herein by reference in their entirety. X may be any bipodal peptide binder comprising a 0-hairpin motif or a leucine-zipper motif as a structure stabilizing region comprising two parallel amino acid strands or two antiparallel amino acid strands, and a target binding region I linked to one terminus of the first of the strands of the structure stabilizing region, and a target binding region II linked to the terminus of the second of the strands of the structure stabilizing region, as disclosed in US Pat. Application No. 20110152500 to Jon et al., the contents of which are incorporated herein by reference in their entirety. X may be any bipodal peptide binder targeting KPI as disclosed in WO2014017743 to Jon et al, any bipodal peptide binder targeting cytokine as disclosed in WO2011132939 to Jon et al., any bipodal peptide binder targeting transcription factor as disclosed in WO201132941 to Jon et al., any bipodal peptide binder targeting G protein-coupled receptor as disclosed in WO2011132938 to Jon et al., any bipodal peptide binder targeting receptor tyrosine kinase as disclosed in WO2011132940 to Jon et al., the contents of each of which are incorporated herein by reference in their entireties. X may also be bipodal peptide binders targeting cluster differentiation (CD7) or an ion channel.

In some embodiments, the target, target cell or marker is a molecule that is present exclusively or predominantly on the surface of malignant cells, e.g., a tumor antigen. In some embodiments, a marker is a prostate cancer marker. In some embodiments the target can be an intra-cellular protein.

In some embodiments, a marker is a breast cancer marker, a colon cancer marker, a rectal cancer marker, a lung cancer marker, a pancreatic cancer marker, a ovarian cancer marker, a bone cancer marker, a renal cancer marker, a liver cancer marker, a neurological cancer marker, a gastric cancer marker, a testicular cancer marker, a head and neck cancer marker, an esophageal cancer marker, or a cervical cancer marker.

The targeting moiety directs the conjugates to specific tissues, cells, or locations in a cell. The target can direct the conjugate in culture or in a whole organism, or both. In each case, the targeting moiety binds to a receptor that is present on the surface of or within the targeted cell(s), wherein the targeting moiety binds to the receptor with an effective specificity, affinity and avidity. In other embodiments the targeting moiety targets the conjugate to a specific tissue such as the liver, kidney, lung or pancreas. The targeting moiety can target the conjugate to a target cell such as a cancer cell, such as a receptor expressed on a cell such as a cancer cell, a matrix tissue, or a protein associated with cancer such as tumor antigen. Alternatively, cells comprising the tumor vasculature may be targeted. Targeting moieties can direct the conjugate to specific types of cells such as specific targeting to hepatocytes in the liver as opposed to Kupffer cells. In other cases, targeting moieties can direct the conjugate to cells of the reticular endothelial or lymphatic system, or to professional phagocytic cells such as macrophages or eosinophils.

In some embodiments the target is member of a class of proteins such as receptor tyrosine kinases (RTK) including the following RTK classes: RTK class I (EGF receptor family) (ErbB family), RTK class II (Insulin receptor family), RTK class III (PDGF receptor family), RTK class IV (FGF receptor family), RTK class V (VEGF receptors family), RTK class VI (HGF receptor family), RTK class VII (Trk receptor family), RTK class VIII (Eph receptor family), RTK class IX (AXL receptor family), RTK class X (LTK receptor family), RTK class XI (TIE receptor family), RTK class XII (ROR receptor family), RTK class XIII (DDR receptor family), RTK class XIV (RET receptor family), RTK class XV (KLG receptor family), RTK class XVI (RYK receptor family) and RTK class XVII (MuSK receptor family).

In some embodiments the target is a serine or threonine kinase, G-protein coupled receptor, methyl CpG binding protein, cell surface glycoprotein, cancer stem cell antigen or marker, carbonic anhydrase, cytolytic T lymphocyte antigen, DNA methyltransferase, an ectoenzyme, a glycosylphosphatidylinositol-anchored co-receptor, a glypican-related integral membrane proteoglycan, a heat shock protein, a hypoxia induced protein, a multi drug resistant transporter, a Tumor-associated macrophage marker, a tumor associated carbohydrate antigen, a TNF receptor family member, a transmembrane protein, a tumor necrosis factor receptor superfamily member, a tumour differentiation antigen, a zinc dependent metallo-exopeptidase, a zinc transporter, a sodium-dependent transmembrane transport protein, a member of the SIGLEC family of lectins, or a matrix metalloproteinase.

Other cell surface markers are useful as potential targets for tumor-homing therapeutics, including, for example HER-2, HER-3, EGFR, and the folate receptor.

In other embodiments, the targeting moiety binds a target such as CD19, CD70, CD56, PSMA, alpha integrin, CD22, CD138, EphA2, AGS-5, Nectin-4, HER2, GPMNB, CD74 and Le.

In some embodiments the target of a target moiety is a protein listed in Category A.

Category A. Non-limiting examples of proteins that may be targeted

| | | | |
|---|---|---|---|
| 5T4 | CD64 | GPIIb/IIIa receptors | PDGFRbeta |
| A20/TNFAIP3 | CD68 | GPR161/RE2 | P-glycoprotein |
| ABCB5 | CD70 | Guanylyl cyclase receptor | Podoplanin |
| | | C | |
| ABCG2 | CD80 | HA-CD44v3 | PON1 |
| AFP | CD86 | HER2/ERBB2 | PRAME |
| ALCAM/CD166 | CD90 | HIF1alpha | PSAM |
| ALDH1A1 | CD96 | HIF-2 | PTEN |
| Apelin J Receptor | CEACAM-5/cd66e | HLA-DR | RAAG12 |
| APN/CD13 | CEACAM-6 | Hsp90 | RON |
| AXL | c-KIT | IGE receptor | sialyl-Le(x) |
| B7H4 | c-Maf | IGF-1R | sialyl-Le(x) |
| BCMA | c-Met | IL-1 alpha | sialyl-Tn |
| BCRP/ABCG2 | Cripto/TDGF-1 | IL-11R | Sigma |
| | | | Receptor/Pgrmc1 |
| BMI-1 | CSFR | IL-1R | SLC34A2 |
| CA9 | CXCR1 | IL-23R | SLC44A4 |
| CAIX | CXCR1 | IL-2R | SLITRK6 |
| mmp | CXCR4 | IL-3 R | SOX2 |
| CanAg | disialylgalactosylgloboside | IL-4R | STAT-3 |
| CD117 | DLL4 | IL-6 R | STEAP-1 |
| CD11a | DNMT1 | Indegrin alpha 6 | STRO-1 |
| CD11b | DNMT3A | iNOS | Tenasin-C |
| CD136 | DNMT3B | Insulin receptor | TF antigen |
| CD138 | DNMT3L | L1CAM | TIM-3 |
| CD14 | EDB (Fibronectin extra | LGR5 | Tissue Factor |
| | domain B) | | (CD142) |
| CD15 | EGFR VIII | LIV-1 (SLC39A6), Zip6 | Tn antigen |
| CD152 (CTLA-4) | E-NPP3/CD203c | LRP | TNFR |
| CD172A | Epcam/TROP1 | MAGE-A3 | TRAIL-R1 |
| CD19 | EphA1 | MBD1 | TRAIL-R2 |
| CD20 | EphA2 | MBD2 | Transferrin receptor |
| CD204 | ERBB3 | MBD4 | TRK-A |
| CD206 | FAP | Mesothelin | TRK-B |
| CD22 | FGFR1 | Metadherin/MTDH/AEG-1 | Trop-2/EGP-1 |
| CD24 | FGFR2 | MICL | UHRF1 |
| CD25 | FGFR3 | MMP-2 | UHRF2 |
| CD26 | FGFR4 | MMP-9 | VEGFR1 |
| CD27 (CD70L) | Fibronectin | MRP1 | VEGFR2 |
| CD28 | Folate receptor | Muc-1 | VEGFR3 |
| CD3 | FRb | MUC16/CA-125 | ZBTB33 |
| CD30 | Galbg4 | Mushai-1 | ZBTB4 |
| CD33 | GD2 ganglioside | NaPi2b | EphA3 |
| CD34 | GD3 ganglioside | Nectin-4 | EphA4 |
| CD38 | GLI-1 | Nestin | EphA5 |
| CD40 | GLI-2 | Neurotensin receptor 1 | EphA6 |
| CD41 | globo-H | NF2 | EphA7 |
| CD44 | GLUT1 | Notch1 | EphA8 |
| CD45 | Glycoprotein NMB | Notch2 | EphB1 |
| CD45.1 | glycosphingolipid P₁ | Notch3 | EphB2 |
| CD45.2 | GM2 ganglioside | Notch4 | EphB3 |
| CD47/IAP | GP130 | Ovastacin | EphB4 |
| CD52 | GPC3 Glypican-3 | PDGFRalpha | EphB5 |
| EphB6 | GRP78 | | |

In some embodiments, the targeting moiety may bind to any human protein.

In certain embodiments, the targeting moiety or moieties of the conjugate are present at a predetermined molar weight percentage from about 1% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the molar weight percentages of the components of the conjugate is 100%. The amount of targeting moieties of the conjugate may also be expressed in terms of proportion to the active agent(s), for example, in a ratio of ligand to active agent of about 10:1, 9:1, 8:1, 7:1, 6:1, 5:1, 4:1, 3:1, 2:1, 1:1, 1:2, 1:3, 1:4; 1:5, 1:6, 1:7, 1:8, 1:9, or 1:10.

### III. Particles and Nanoparticles

Particles comprising one or more conjugates can be polymeric particles, lipid particles, solid lipid particles, inorganic particles, or combinations thereof (e.g., lipid stabilized polymeric particles). In some embodiments, the conjugates are substantially encapsulated or particularly encapsulated in the particles. In some embodiments, the conjugates are disposed on the surface of the particles. The conjugates may be attached to the surface of the particles with covalent bonds, or non-covalent interactions. In some embodiments, the conjugates of the present invention self-assemble into a particle.

As used herein, the term “encapsulate” means to enclose, surround or encase. As it relates to the formulation of the conjugates of the invention, encapsulation may be substantial, complete or partial. The term “substantially encapsulated” means that at least greater than 50, 60, 70, 80, 85, 90, 95, 96, 97, 98, 99, 99.9, 99.99 or greater than 99.999% of conjugate of the invention may be enclosed, surrounded or encased within the particle. “Partially encapsulation” means that less than 10, 10, 20, 30, 40 50 or less of the conjugate of the invention may be enclosed, surrounded or encased within the particle. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the particle. Encapsulation may be determined by any known method. In some embodiments, the particles are polymeric particles or contain a polymeric matrix. The particles can contain any of the polymers described herein or derivatives or copolymers thereof. The particles will generally contain one or more biocompatible polymers. The polymers can be biodegradable polymers. The polymers can be hydrophobic polymers, hydrophilic polymers, or amphiphilic polymers. In some embodiments, the particles contain one or more polymers having an additional targeting moiety attached thereto. In some embodiments, the particles are inorganic particles, such as but not limited to, gold nanoparticles and iron oxide nanoparticles.

The size of the particles can be adjusted for the intended application. The particles can be nanoparticles or microparticles. The particle can have a diameter of about 10 nm to about 10 microns, about 10 nm to about 1 micron, about 10 nm to about 500 nm, about 20 nm to about 500 nm, or about 25 nm to about 250 nm. In some embodiments the particle is a nanoparticle having a diameter from about 25 nm to about 250 nm. In some embodiments, the particle is a nanoparticle having a diameter from about 50 nm to about 150 nm. In some embodiments, the particle is a nanoparticle having a diameter from about 70 nm to about 130 nm. In some embodiments, the particle is a nanoparticle having a diameter of about 100 nm. It is understood by those in the art that a plurality of particles will have a range of sizes and the diameter is understood to be the median diameter of the particle size distribution. Polydispersity index (PDI) of the particles may be ≤ about 0.5, ≤ about 0.2, or ≤ about 0.1. In some embodiments, the nanoparticles have low PDI but bimodal distribution. Drug loading may be ≥ about 1%, ≥ about 5%, ≥ about 10%, or ≥ out 20%. Drug loading, as used herein, refers to the weight ratio of the conjugates of the invention and depends on maximum tolerated dose (MTD) of free drug conjugate. Particle ζ-potential (in 1/10^{th} PBS) may be ≤0 mV or from about −10 to 0 mV. Drug released in vitro from the particle at 2h may be less than about 60%, less than about 40%, or less than about 20%. Regarding pharmacokinetics, plasma area under the curve (AUC) in a plot of concentration of drug in blood plasma against time may be at least 2 fold greater than free drug conjugate, at least 4 fold greater than free drug conjugate, at least 5 fold greater than free drug conjugate, at least 8 fold greater than free drug conjugate, or at least 10 fold greater than free drug conjugate. Tumor PK/PD of the particle may be at least 5 fold greater than free drug conjugate, at least 8 fold greater than free drug conjugate, at least 10 fold greater than free drug conjugate, or at least 15 fold greater than free drug conjugate. The ratio of Cₘₐₓ of the particle to Cₘₐₓ of free drug conjugate may be at least about 2, at least about 4, at least about 5, or at least about 10. Cₘₐₓ, as used herein, refers to the maximum or peak serum concentration that a drug achieves in a specified compartment or test area of the body after the drug has been administrated and prior to the administration of a second dose. The ratio of MTD of a particle to MTD of free drug conjugate may be at least about 0.5, at least about 1, at least about 2, or at least about 5. Efficacy in tumor models, e.g., TGI %, or modulation of pharmacodynamics biomarkers (e.g. higher intensity, temporal profile) of a particle is better than free drug conjugate. Toxicity of a particle is lower than free drug conjugate.

In various embodiments, a particle may be a nanoparticle, i.e., the particle has a characteristic dimension of less than about 1 micrometer, where the characteristic dimension of a particle is the diameter of a perfect sphere having the same volume as the particle. The plurality of particles can be characterized by an average diameter (e.g., the average diameter for the plurality of particles). In some embodiments, the diameter of the particles may have a Gaussian-type distribution. In some embodiments, the plurality of particles have an average diameter of less than about 300 nm, less than about 250 nm, less than about 200 nm, less than about 150 nm, less than about 100 nm, less than about 50 nm, less than about 30 nm, less than about 10 nm, less than about 3 nm, or less than about 1 nm. In some embodiments, the particles have an average diameter of at least about 5 nm, at least about 10 nm, at least about 30 nm, at least about 50 nm, at least about 100 nm, at least about 150 nm, or greater. In certain embodiments, the plurality of the particles have an average diameter of about 10 nm, about 25 nm, about 50 nm, about 100 nm, about 150 nm, about 200 nm, about 250 nm, about 300 nm, about 500 nm, or the like. In some embodiments, the plurality of particles have an average diameter between about 10 nm and about 500 nm, between about 50 nm and about 400 nm, between about 100 nm and about 300 nm, between about 150 nm and about 250 nm, between about 175 nm and about 225 nm, or the like. In some embodiments, the plurality of particles have an average diameter between about 10 nm and about 500 nm, between about 20 nm and about 400 nm, between about 30 nm and about 300 nm, between about 40 nm and about 200 nm, between about 50 nm and about 175 nm, between about 60 nm and about 150 nm, between about 70 nm and about 130 nm, or the like. For example, the average diameter can be between about 70 nm and 130 nm. In some embodiments, the plurality of particles have an average diameter between about 20 nm and about 220 nm, between about 30 nm and about 200 nm, between about 40 nm and about 180 nm, between about 50 nm and about 170 nm, between about 60 nm and about 150 nm, or between about 70 nm and about 130 nm. In one embodiment, the particles have a size of 40 to 120 nm with a zeta potential close to 0 mV at low to zero ionic strengths (1 to 10 mM), with zeta potential values between +5 to −5 mV, and a zero/neutral or a small −ve surface charge.

### A. Polymers

The particles may contain one or more polymers. Polymers may contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(ε-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker.

The particles may contain one or more hydrophilic polymers. Hydrophilic polymers include cellulosic polymers such as starch and polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol); polyoxazoline; and copolymers thereof.

The particles may contain one or more hydrophobic polymers. Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In certain embodiments, the hydrophobic polymer is an aliphatic polyester. In some embodiments, the hydrophobic polymer is poly(lactic acid), poly(glycolic acid), or poly(lactic acid-co-glycolic acid).

In some embodiments, the particles may comprise triblock copolymers that self assemble and complex with the conjugates. Such triblock copolymers may comprise spatially separated hydrophobic and hydrophilic parts that have been developed for the effective delivery of negatively charged molecules such as nucleic acids including siRNAs. In one embodiment, the triblock copolymer may comprise a hydrophilic block, a hydrophobic block, and a positively charged block capable of reversibly complexing a negatively charged molecule. Any triblock copolymer disclosed in US 20100222407 to Segura et al., the contents of which are incorporated herein by reference in their entirety, may be used to complex with conjugates of the present invention and self assemble into a supramolecular structure such particles.

The particles can contain one or more biodegradable polymers. Biodegradable polymers can include polymers that are insoluble or sparingly soluble in water that are converted chemically or enzymatically in the body into water-soluble materials. Biodegradable polymers can include soluble polymers crosslinked by hydolyzable cross-linking groups to render the crosslinked polymer insoluble or sparingly soluble in water.

Biodegradable polymers in the particle can include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose such as methyl cellulose and ethyl cellulose, hydroxyalkyl celluloses such as hydroxypropyl cellulose, hydroxy-propyl methyl cellulose, and hydroxybutyl methyl cellulose, cellulose ethers, cellulose esters, nitro celluloses, cellulose acetate, cellulose propionate, cellulose acetate butyrate, cellulose acetate phthalate, carboxylethyl cellulose, cellulose triacetate, cellulose sulphate sodium salt, polymers of acrylic and methacrylic esters such as poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene imines), poly(caprolactones), poly(hydroxyalkanoates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. In some embodiments the particle contains biodegradable polyesters or polyanhydrides such as poly(lactic acid), poly(glycolic acid), and poly(lactic-co-glycolic acid).

The particles can contain one or more amphiphilic polymers. Amphiphilic polymers can be polymers containing a hydrophobic polymer block and a hydrophilic polymer block. The hydrophobic polymer block can contain one or more of the hydrophobic polymers above or a derivative or copolymer thereof. The hydrophilic polymer block can contain one or more of the hydrophilic polymers above or a derivative or copolymer thereof. In some embodiments the amphiphilic polymer is a di-block polymer containing a hydrophobic end formed from a hydrophobic polymer and a hydrophilic end formed of a hydrophilic polymer. In some embodiments, a moiety can be attached to the hydrophobic end, to the hydrophilic end, or both. The particle can contain two or more amphiphilic polymers.

### B. Lipids

The particles may contain one or more lipids or amphiphilic compounds. For example, the particles can be liposomes, lipid micelles, solid lipid particles, or lipid-stabilized polymeric particles. The lipid particle can be made from one or a mixture of different lipids. Lipid particles are formed from one or more lipids, which can be neutral, anionic, or cationic at physiologic pH. The lipid particle is preferably made from one or more biocompatible lipids. The lipid particles may be formed from a combination of more than one lipid, for example, a charged lipid may be combined with a lipid that is non-ionic or uncharged at physiological pH.

The particle can be a lipid micelle. Lipid micelles for drug delivery are known in the art. Lipid micelles can be formed, for instance, as a water-in-oil emulsion with a lipid surfactant. An emulsion is a blend of two immiscible phases wherein a surfactant is added to stabilize the dispersed droplets. In some embodiments the lipid micelle is a microemulsion. A microemulsion is a thermodynamically stable system composed of at least water, oil and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents.

The particle can be a liposome. Liposomes are small vesicles composed of an aqueous medium surrounded by lipids arranged in spherical bilayers. Liposomes can be classified as small unilamellar vesicles, large unilamellar vesicles, or multi-lamellar vesicles. Multi-lamellar liposomes contain multiple concentric lipid bilayers. Liposomes can be used to encapsulate agents, by trapping hydrophilic agents in the aqueous interior or between bilayers, or by trapping hydrophobic agents within the bilayer.

The lipid micelles and liposomes typically have an aqueous center. The aqueous center can contain water or a mixture of water and alcohol. Suitable alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

The particle can be a solid lipid particle. Solid lipid particles present an alternative to the colloidal micelles and liposomes. Solid lipid particles are typically submicron in size, i.e. from about 10 nm to about 1 micron, from 10 nm to about 500 nm, or from 10 nm to about 250 nm. Solid lipid particles are formed of lipids that are solids at room temperature. They are derived from oil-in-water emulsions, by replacing the liquid oil by a solid lipid.

Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-a-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

Suitable cationic lipids include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N—(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

Suitable solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, preferably 12-30 carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Suitable solid lipids can include cetyl palmitate, beeswax, or cyclodextrin.

Amphiphilic compounds include, but are not limited to, phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), for example, between 0.1-30 (weight lipid/w polymer). Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

### C. Hydrophobic Ion-Pairing Complexes

The particles may comprise hydrophobic ion-pairing complexes or hydrophobic ioin-pairs formed by one or more conjugates described above and counterions.

Hydrophobic ion-pairing (HIP) is the interaction between a pair of oppositely charged ions held together by Coulombic attraction. HIP, as used here in, refers to the interaction between the conjugate of the present invention and its counterions, wherein the counterion is not H⁺ or HO⁻ ions. Hydrophobic ion-pairing complex or hydrophobic ion-pair, as used herein, refers to the complex formed by the conjugate of the present invention and its counterions. In some embodiments, the counterions are hydrophobic. In some embodiments, the counterions are provided by a hydrophobic acid or a salt of a hydrophobic acid. In some embodiments, the counterions are provided by bile acids or salts, fatty acids or salts, lipids, or amino acids. In some embodiments, the counterions are negatively charged (anionic). Non-limited examples of negative charged counterions include the counterions sodium sulfosuccinate (AOT), sodium oleate, sodium dodecyl sulfate (SDS), human serum albumin (HSA), dextran sulphate, sodium deoxycholate, sodium cholate, anionic lipids, amino acids, or any combination thereof. Non-limited examples of positively charged counterions include 1,2-dioleoyl-3-trimethylammonium-propane (chloride salt) (DOTAP), cetrimonium bromide (CTAB), quaternary ammonium salt didodecyl dimethylammonium bromide (DMAB) or Didodecyldimethylammonium bromide (DDAB). Without wishing to be bound by any theory, in some embodiments, HIP may increase the hydrophobicity and/or lipophilicity of the conjugate of the present invention. In some embodiments, increasing the hydrophobicity and/or lipophilicity of the conjugate of the present invention may be beneficial for particle formulations and may provide higher solubility of the conjugate of the present invention in organic solvents. Without wishing to be bound by any theory, it is believed that particle formulations that include HIP pairs have improved formulation properties, such as drug loading and/or release profile. Without wishing to be bound by any theory, in some embodiments, slow release of the conjugate of the invention from the particles may occur, due to a decrease in the conjugate's solubility in aqueous solution. In addition, without wishing to be bound by any theory, complexing the conjugate with large hydrophobic counterions may slow diffusion of the conjugate within a polymeric matrix. In some emobodiments, HIP occurs without covalent conjugation of the counterion to the conjugate of the present invention.

Without wishing to be bound by any theory, the strength of HIP may impact the drug load and release rate of the particles of the invention. In some embodiments, the strength of the HIP may be increased by increasing the magnitude of the difference between the pKa of the conjugate of the present invention and the pKa of the agent providing the counterion. Also without wishing to be bound by any theory, the conditions for ion pair formation may impact the drug load and release rate of the particles of the invention.

In some embodiments, any suitable hydrophobic acid or a combination thereof may form a HIP pair with the conjugate of the present invention. In some embodiments, the hydrophobic acid may be a carboxylic acid (such as but not limited to a monocarboxylic acid, dicarboxylic acid, tricarboxylic acid), a sulfinic acid, a sulfenic acid, or a sulfonic acid. In some embodiments, a salt of a suitable hydrophobic acid or a combination thereof may be used to form a HIP pair with the conjugate of the present invention. Examples of hydrophobic acids, saturated fatty acids, unsaturated fatty acids, aromatic acids, bile acid, polyelectrolyte, their dissociation constant in water (pKa) and log P values were disclosed in WO2014/043,625, the content of which is incorporated herein by reference in its entirety. The strength of the hydrophobic acid, the difference between the pKa of the hydrophobic acid and the pKa of the conjugate of the present invention, log P of the hydrophobic acid, the phase transition temperature of the hydrophobic acid, the molar ratio of the hydrophobic acid to the conjugate of the present invention, and the concentration of the hydrophobic acid were also disclosed in WO2014/043,625, the content of which is incorporated herein by reference in its entirety.

In some embodiments, particles of the present invention comprising a HIP complex and/or prepared by a process that provides a counterion to form HIP complex with the conjugate may have a higher drug loading than particles without a HIP complex or prepared by a process that does not provide any counterion to form HIP complex with the conjugate. In some embodiments, drug loading may increase 50%, 100%, 2 times, 3 times, 4 times, 5 times, 6 times, 7 times, 8 times, 9 times, or 10 times.

In some embodiments, the particles of the invention may retain the conjugate for at least about 1 minute, at least about 15 minutes, at least about 1 hour, when placed in a phosphate buffer solution at 37° C.

### D. Additional Targeting Moieties

The particles can contain one or more targeting moieties targeting the particle to a specific organ, tissue, cell type, or subcellular compartment in addition to the targeting moieties of the conjugate. The additional targeting moieties can be present on the surface of the particle, on the interior of the particle, or both. The additional targeting moieties can be immobilized on the surface of the particle, e.g., can be covalently attached to polymer or lipid in the particle. In preferred embodiments, the additional targeting moieties are covalently attached to an amphiphilic polymer or a lipid such that the targeting moieties are oriented on the surface of the particle.

### E. Nonaparticles Comprising Components of the CRISPR-Cas System

In some embodiments, nanoparticles of the present invention may comprise one or more conjugats of the present invention with the formula of (sgRNA)n-linker-Targeting Moiety as described above; and one or more Cas proteins. The conjugates comprising sgRNAs associated with the CRISPR-Cas system may be any conjugate described herein. The Cas proteins packaged in the present nanoparticle may be a polypeptide, a mRNA molecule that encodes the Cas protein, or an expression construct that comprising a nucleic acid molecule encoding the Cas protein, or a vector including such an expression construct.

In some embodiments, the Cas protein is a type II CRISPR Cas9 endonuclease. The Cas9 nuclease may be a wild type Cas9, a nickase with only one active nuclease domain, a catalytically inactive dCas9, a Cas9 homolog or ortholog, or a fusion protein comprising the dCas9 protein fused with a heterogeneous effector domain of a particular function.

In some examples, the Cas9 nuclease may be derived from S. pneumoniae, Streptococcus pyogenes, Streptococcus thermophilus, Neisseria Meningitidis or functional variants thereof (US Patent Publication No. 20140399405 to Sontheimer, the content of which is incorporated by reference in its entirety).

In some examples, the Cas9 protein may be a Cas9 nickase that has a catalytically inactive nuclease domain bearing mutations selected from selected from D10A, H840A, N854A, and N863A (as for Cas9 from S. pyogenes).

In some examples, A Cas nuclease may be a catalytically inactive dCas9 protein. The catalytic inactivity is considered to substantially lack all DNA cleavage activity when the DNA cleavage activity of the mutated nuclease is less than about 25%, 10%, 5%, 1%, 0.1%, 0.01%, or lower with respect to its non-mutated form. For the Cas9 protein from S. pyogenes, any or all of the following mutations are included: D10A, E762A, H840A, N854A, N863A and/or D986A; as well as other conservative substitutions for any of the replacement amino acids. The same (or conservative substitutions of these mutations) at corresponding positions 10, 762, 840, 854, 863 and/or 986 of Cas9 (S. pyogenes) in other Cas9 proteins are also included.

In some examples, the Cas9 protein may be a Cas9 ortholog identified in any bacteria strains as listed previously in the present disclosure, or functional variants thereof.

In some examples, the Cas9 protein may be codon-optimized which bears a C-terminus SV40 nuclear localization signal. Cas9 homologs with higher specificity may be included in the present nanoparticles.

In some embodiments, the Cas9 protein may be a dual enzyme. In one example, the catalytically inactive Cas9 (dCas9) protein is fused with the dimerization-dependent Fokl nuclease domain. Such Dimerizaton may increase the specificity of RNA-guided nucleases. In this system, sequence-specific DNA cleavage only occurs upon dimerization of two Fokl nuclease domains from two different RNA-guided Fokl nucleases (RFNs) that are bound in close proximity to two unique target sites (Tsai et al., Nat Biotechnol. 2014; 32:569-576; and Guilinger et al., Nat Biotechnol. 2014; 32:577).

In some embodiments, the Cas9 nuclease, or functional variants thereof, is compelxed with conjugates comprising one or more sgRNA molecules.

In some embodiments, other Cas proteins may be included in the present nanoparticles. In some examples, the Cas protein is a core Cas protein. Exemplary Cas core proteins include, but are not limited to Cas1, Cas2, Cas3, Cas4, Cas5, Cash, Cas7, Cas8 and Cas9. A Cas protein may be a Cas protein of the Dvulg subtype (also known as CASS1) including, but not limited to Csd1, Csd2, and Cas5d; or a Cas protein of an E. coli subtype (also known as CASS2), including but not limited to Cse1, Cse2, Cse3, Cse4, and Cas5e; or a Cas protein of Ypest subtype (also known as CASS3), including but not limited to, Csy2, Csy3, and Csy4; or a Cas protein of the Nmeni subtype (also known as CASS4) including but not limited to Csn1 and Csn2; or a Cas protein of the Apern subtype (also known as CASS5) including, but not limited to Csa1, Csa2, Csa3, Csa4, Csa5, and Cas5a; or a Cas protein of the Mtube subtype (also known as CASS6) including, but not limited to Csm1, Csm2, Csm3, Csm4, and Csm5; or a Cas protein of the Tneap subtype (also known as CASS7) including, but not limited to, Cst1, Cst2, Cas5t; or a Cas protein of the Hmari subtype including, but not limited to Csh1, Csh2, and Cas5h; or a Cas protein comprising a RAMP module Cas protein including, but not limited to, Cmr1, Cmr2, Cmr3, Cmr4, Cmr5, and Cmr6.

In some embodiments, the Cas protein (e.g., Cas9) may be conjugated to or fused to a cell-penetrating polypeptide, a charged protein that carries a positive, negative or overall neutral electric charge (e.g., superpositively charged GFP), or a protein transduction domain (PTD) (e.g., Tat, oliogarginine and penetratin) to increase its entry to a cell. As Used herein the term “cell penetrating peptide” refers to a polypeptide or peptide, respectively, which facilitates the uptake of molecule into a cell.

In some embodiments, the Cas protein included in the present nanoparticles may be in the form of a nucleic acid molecule that encodes the Cas protein (e.g., Cas9). The nucleic acid molecule encoding the Cas protein may a DNA or RNA molecule, such as a messenger RNA (mRNA), a DNA construct comprising the polynucleotide encoding the Cas protein or a vector that contains such a construct.

In some embodiments, the nucleic acid may be a mRNA encoding the Cas protein. In other examples, the Cas9 protein may be encoded by a modified mRNA. The modified mRNA molecule may be modified to prevent rapid degradation by endo- and exo-nucleases and to avoid or reduce the cell's innate immune or interferon response to the mRNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. Non-limiting examples of modified internucleoside linkages include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs. Other internucleoside linkages may not include a phosphorus atom, but are formed by short chain alkyl or cycloalkyl internucleoside linkages, mixed heteroatoms and alkyl or cycloalkyl internucleoside linkages, or one or more short chain heteroatomic or heterocyclic internucleoside linkages. Some patents that tach such modifications include but are not llimited to U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; and 5,677,437; each of which is herein incorporated by reference in its entirety.

In some embodiments, the modified mRNA encoding a Cas protein may also contain one or more substituted sugar moieties, such as 2′ methoxyethoxy (2′-O—CH2CH2OCH3, also known as 2′-O-(2-methoxyethyl) or 2′-MOE); 2′-dimethylaminooxyethoxy; 2′-methoxy (2′-OCH3), 2′-aminopropoxy (2′-OCH2CH2CH2NH2) and 2′-fluoro (2′-F). Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

In some embodiments, the modified mRNA encoding a Cas protein may comprise at least one modified nucleoside including but not limited to 5-methylcytidine (5mC), N6-methyladenosine (m6A), 3,2′-O-dimethyluridine (m4U), 2-thiouridine (s2U), 2′ fluorouridine, pseudouridine, 2′-O-methyluridine (Urn), 2′ deoxyuridine (2′ dU), 4-thiouridine (s4U), 5-methyluridine (m5U), 2′-O-methyladenosine (m6A), N6,2′-O-dimethyladenosine (m6Am), N6,N6,2′-O-trimethyladenosine (m62Am), 2′-O-methylcytidine (Cm), 7-methylguanosine (m7G), 2′-O-methylguanosine (Gm), N2,7-dimethylguanosine (m2,7G), N2,N2,7-trimethylguanosine (m2,2,7G), and inosine (I). In other embodiments, the modified mRNA encoding a Cas protein may contain other naturally-occurred or synthetic nucleoside substitutes. Exemplary nucleobases may include, but are not limited to, inosine, xanthine, hypoxanthine, nubularine, isoguanisine, tubercidine, 2-(halo)adenine, 2-(alkyl)adenine, 2-(propyl)adenine, 2 (amino)adenine, 2-(aminoalkyl)adenine, 2 (aminopropyl)adenine, 2 (methylthio) N6 (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl)adenine, 8-(thiol)adenine, N6-(isopentyl)adenine, N6 (methyl)adenine, N6,N6 (dimethyl)adenine, 2-(alkyl)guanine, 2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N (methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, 5 (halo)cytosine, 5 (methyl)cytosine, 5 (propynyl)cytosine, 5 (propynyl)cytosine, 5 (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, 5 (methyl) 2,4 (dithio)uracil, 5 (methylaminomethyl)-2,4 (dithio)uracil, 5 (2-aminopropyl)uracil, 5-(alkyl)uracil, 5-(alkynyOuracil, 5-(allylamino)uracil, 5 (aminoallyl)uracil, 5 (aminoalkyl)uracil, 5 (guanidiniumalkyl)uracil, 5 (1,3-diazole-1-alkyl)uracil, 5-(cyanoalkyOuracil, 5-(dialkylaminoalkyOuracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, 5 (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil, 4 (thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 (aminocarbonylethylenyl)-2,4-(dithio)pseudouracil, 1) (aminoalkylaminocarbonylethylenyl)-pseudouracil, 1 (aminoalkylamino-carbonylethylenyl)-2(thio)-pseudouracil, 1 (aminoalkylaminocarbonylethylenyl)-4 (thio)pseudouracil, 1 (aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkylhydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynypisocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenzyl, tetracenyl, pentacenyl, difluorotolyl, 4-(fluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino)purine, 5 substituted pyrimidines, N2-substituted purines, N6-substituted purines, 06-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Modified nucleosides also include natural bases that comprise conjugated moieties, e.g. a ligand. Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference in its entirety.

In some embodiments, the modified mRNA encoding a Cas protein may further comprise 5′cap, e.g., a modified guanine nucleotide that is linked to the 5′ end of an RNA molecule using a 5′-5′ triphosphate linkage; 5′ diguanosine cap, tetraphosphate cap analogs having a methylene-bis(phosphonate) moiety (see e.g., Rydzik, A M et al., (2009) Org Biomol Chem 7(22):4763-76), dinucleotide cap analogs having a phosphorothioate modification (see e.g., Kowalska, J. et al., (2008) RNA 14(6):1119-1131), cap analogs having a sulfur substitution for a non-bridging oxygen (see e.g., Grudzien-Nogalska, E. et al., (2007) RNA 13(10): 1745-1755), N7-benzylated dinucleoside tetraphosphate analogs (see e.g., Grudzien, E. et al., (2004) RNA 10(9):1479-1487), or anti-reverse cap analogs (see e.g., Jemielity, J. et al., (2003) RNA 9(9): 1108-1.122).

In some embodiments, the modified mRNA encoding a Cas protein may comprise a 5′ and/or 3′ untranslated region (UTR); and/or other signal nucleotide sequence (e.g. nuclear localization signal NLS). In some examples, it may comprise one or more nuclear localization signals, such as about 1, 2, 3, 4, 5, 6, 7, 8, 9, 10, or more NLS, either at or near the amino-terminus, or at or near the carboxyl terminus. The one or more NLSs are of sufficient strength to drive accumulation of the Cas9 protein in a detectable amount in the nucleus of a eukaryotic cell.

In some embodiments, the nucleic acid molecule encoding a Cas protein may be codon-optimized for expression in particular cells, such as eukaryotic cells (e.g., mammalian cells including but not limited to human, non-human primate, mouse, rat, rabbit, or dog). As used herein, the term “codon-optimization” refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence.

In some embodiments, a Cas protein included in nanoparticles of the present invention may be a Cas9 fusion protein comprising a catalytically inactive Cas 9 (dCas9) protein fused with one or more heterogeneous effector domains. As used herein, the term “heterogeneous effector domain” means a heterogeneous protein domain that can perform a distinct regulatory function to a genomic nucleic acid sequence, for instance, a protein domain that can 1). affect either transcriptional repression or activation of a gene(s), e.g., a transcriptional activation domain derived from VP64 or NF-κB p65; 2). catalytically modify histones, e.g., histone acetyltransferases (HAT), histone deacetylases (HDAC), or histone demethylases; or 3). catalytically chemically modify DNA, e.g., DNA methyltransferase (DNMT) or TET proteins that modify the methylation state of DNA. In this context, the dCas9 protein serves as a platform to guide a heterogeneous effector to a target polynucleotide determined by the sequence specificity of the sgRNA molecule.

Additionally, the dCas9 protein can be fused with other nuclease domains such as Fokl to enable ‘highly specific’ genome editing contingent upon dimerization of nuadjacent sgRNA-Cas9 complexes; or with fluorescent proteins for visualizing genomic loci and chromosome dynamics; or with other fluorescent molecules such as protein or nucleic acid bound organic fluorophores, quantum dots, molecular beacons and echo probes or molecular beacon replacements; or with multivalent ligand-binding protein domains that enable programmable manipulation of genome-wide 3D architecture.

Other additional protein sequences that may be fused with the dCas9 protein, optionally through a linker sequence between any two domains, may include, but are not limited to, epitope tags such as histidine (His) tags, V5 tags, FLAG tags, influenza hemagglutinin (HA) tags, Myc tags, VSV-G tags, and thioredoxin (Trx) tags; reporter genes such as glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). Additional domains that may form part of a dCas9 fusion protein are described in US20110059502, the content of which is incorporated herein by reference in its entirety.

In some embodiments, the dCas9 protein comprises mutations at D10A and H840A. In some embodiments, the heterologous effector domain is linked to the N terminus or C terminus of the catalytically inactive dCas9 protein, with an optional intervening linker, wherein the linker does not interfere with activity of the fusion protein. In some embodiments, the dCas9-effector fusion protein may be provided as a fusion nucleic acid molecule encoding the fusion proteins described herein, or a vector including such nucleic acid molecules.

Some exemplary chimeric fusion proteins comprising dCas9 and a heterogeneous effector domain are described in US patent publication NOs.: 20150191744; 20150044772; 20140377868; each of which is incorporated by reference herein in their entirety.

In some embodiments, nanoparticles of the present invention may comprise one or more conjugats of the present invention with the formula of (sgRNA)n-linker-Targeting Moiety as described above; and one or more Cas proteins fragments. The fragments are peptides or polypeptides comprising about 20 to about 300, preferably about 50 to about 250 amino acids. The fragments may self assemble into folded Cas proteins spontaneously. Alternatively, the fragements may be inducible systems, wherein an inducer is conjugated to the sgRNA, incorporated in the nanoparticles or mixed with the nanoparticles.

### IV. Formulations

In some embodiments, conjugates, particles of the present invention may be formulated as liquid suspensions or as freeze-dried products. Suitable liquid preparations may include, but are not limited to, isotonic aqueous solutions, suspensions, emulsions, or viscous compositions that are buffered to a selected pH.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit. As used herein, the term “active ingredient” refers to any chemical and biological substance that has a physiological effect in human or in animals, when exposed to it. In the context of the present invention, the active ingredient in the formulations may be any conjugates and particles as discussed herein above.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The conjugates or particles of the present invention can be formulated using one or more excipients to: (1) increase stability; (2) permit the sustained or delayed release (e.g., from a depot formulation of the monomaleimide); (3) alter the biodistribution (e.g., target the monomaleimide compounds to specific tissues or cell types); (4) alter the release profile of the monomaleimide compounds in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present invention may also include, without limitation, lipidoids, liposomes, lipid nanoparticles, polymers, lipoplexes, core-shell nanoparticles, peptides, proteins, hyaluronidase, nanoparticle mimics and combinations thereof. Accordingly, the formulations of the invention may include one or more excipients, each in an amount that together increases the stability of the monomaleimide compounds.

In some embodiments, the conjugates or particles of the present invention are formulated in aqueous formulations such as pH 7.4 phosphate-buffered formulation, or pH 6.2 citrate-buffered formulation; formulations for lyophilization such as pH 6.2 citrate-buffered formulation with 3% mannitol, pH 6.2 citrate-buffered formulation with 4% mannitol/1% sucrose; or a formulation prepared by the process disclosed in U.S. Pat. No. 8,883,737 to Reddy et al. (Endocyte), the contents of which are incorporated herein by reference in their entirety.

In some embodiments, the conjugates or particles of the present invention targets folate receptors and are formulated in liposomes prepared following methods by Leamon et al. in Bioconjugate Chemistry, vol. 14 738-747 (2003), the contents of which are incorporated herein by reference in their entirety. Briefly, folate-targeted liposomes will consist of 40 mole % cholesterol, either 4 mole % or 6 mole % polyethylene glycol (Mr^{˜}2000)-derivatized phosphatidylethanolamine (PEG2000-PE, Nektar, Ala., Huntsville, Ala.), either 0.03 mole % or 0.1 mole % folate-cysteine-PEG3400-PE and the remaining mole % will be composed of egg phosphatidylcholine, as disclosed in U.S. Pat. No. 8,765,096 to Leamon et al. (Endocyte), the contents of which are incorporated herein by reference in their entirety. Lipids in chloroform will be dried to a thin film by rotary evaporation and then rehydrated in PBS containing the drug. Rehydration will be accomplished by vigorous vortexing followed by 10 cycles of freezing and thawing. Liposomes will be extruded 10 times through a 50 nm pore size polycarbonate membrane using a high-pressure extruder. Similarly, liposomes not targeting folate receptors may be prepared identically with the absence of folate-cysteine-PEG3400-PE.

In some embodiments, the conjugates or particles of the present invention are formulated in parenteral dosage forms including but limited to aqueous solutions of the conjugates or particles, in an isotonic saline, 5% glucose or other pharmaceutically acceptable liquid carriers such as liquid alcohols, glycols, esters, and amides, as disclosed in U.S. Pat. No. 7,910,594 to Vlahov et al. (Endocyte), the contents of which are incorporated herein by reference in their entirety. The parenteral dosage form may be in the form of a reconstitutable lyophilizate comprising the dose of the conjugates or particles. Any prolonged release dosage forms known in the art can be utilized such as, for example, the biodegradable carbohydrate matrices described in U.S. Pat. Nos. 4,713,249; 5,266,333; and 5,417,982, the disclosures of which are incorporated herein by reference, or, alternatively, a slow pump (e.g., an osmotic pump) can be used.

In some embodiments, the parenteral formulations are aqueous solutions containing carriers or excipients such as salts, carbohydrates and buffering agents (e.g., at a pH of from 3 to 9). In some embodiments, the conjugates or particles of the present invention may be formulated as a sterile non-aqueous solution or as a dried form and may be used in conjunction with a suitable vehicle such as sterile, pyrogen-free water. The preparation of parenteral formulations under sterile conditions, for example, by lyophilization under sterile conditions, may readily be accomplished using standard pharmaceutical techniques well-known to those skilled in the art. The solubility of a conjugates or particles used in the preparation of a parenteral formulation may be increased by the use of appropriate formulation techniques, such as the incorporation of solubility-enhancing agents.

In some embodiments, the conjugates or particles of the present invention may be prepared in an aqueous sterile liquid formulation comprising monobasic sodium phosphate monohydrate, dibasic disodium phosphate dihydrate, sodium chloride, potassium chloride and water for injection, as disclosed in US 20140140925 to Leamon et al., the contents of which are incorporated herein by reference in their entirety. For example, the conjugates or particles of the present invention may be formulated in an aqueous liquid of pH 7.4, phosphate buffered formulation for intravenous administration as disclosed in Example 23 of WO2011014821 to Leamon et al. (Endocyte), the contents of which are incorporated herein by reference in their entirety. According to Leamon, the aqueous formulation needs to be stored in the frozen state to ensure its stability.

In some embodiments, the conjugates or particles of the present invention are formulated for intravenous (IV) administration. Any formulation or any formulation prepared according to the process disclosed in US 20140030321 to Ritter et al. (Endocyte), the contents of which are incorporated herein by reference in their entirety, may be used. For example, the conjugates or particles may be formulated in an aqueous sterile liquid formulation of pH 7.4 phosphate buffered composition comprising sodium phosphate, monobasic monohydrate, disodium phosphate, dibasic dehydrate, sodium chloride, and water for injection. As another example, the conjugates or particles may be formulated in pH 6.2 citrated-buffered formulation comprising trisodium citrate, dehydrate, citric acid and water for injection. As another example, the conjugates or particles may be formulated with 3% mannitol in a pH 6.2 citrate-buffered formulation for lyophilization comprising trisodium citrate, dehydrate, citric acid and mannitol. 3% mannitol may be replaced with 4% mannitol and 1% sucrose.

In some embodiments, the particles comprise biocompatible polymers. In some embodiments, the particles comprise about 0.2 to about 35 weight percent of a therapeutic agent; and about 10 to about 99 weight percent of a biocompatible polymer such as a diblock poly(lactic) acid-poly(ethylene)glycol as disclosed in US 20140356444 to Troiano et al. (BIND Therapeutics), the contents of which are incorporated herein by reference in their entirety. Any therapeutically particle composition in U.S. Pat. Nos. 8,663,700, 8,652,528, 8,609,142, 8,293,276 and 8,420,123, the contents of each of which are incorporated herein by reference in their entirety, may also be used.

In some embodiments, the particles comprise a hydrophobic acid. In some embodiments, the particles comprise about 0.05 to about 30 weight percent of a substantially hydrophobic acid; about 0.2 to about 20 weight percent of a basic therapeutic agent having a protonatable nitrogen; wherein the pKa of the basic therapeutic agent is at least about 1.0 pKa units greater than the pKa of the hydrophobic acid; and about 50 to about 99.75 weight percent of a diblock poly(lactic) acid-poly(ethylene)glycol copolymer or a diblock poly(lactic acid-co-glycolic acid)-poly(ethylene)glycol copolymer, wherein the therapeutic nanoparticle comprises about 10 to about 30 weight percent poly(ethylene)glycol as disclosed in WO2014043625 to Figueiredo et al. (BIND Therapeutics), the contents of which are incorporated herein by reference in their entirety. Any therapeutic particle composition in US 20140149158, 20140248358, 20140178475 to Figueiredo et al., the contents of each of which are incorporated herein by reference in their entirety, may also be used.

In some embodiments, the particles comprise a chemotherapeutic agent; a diblock copolymer of poly(ethylene)glycol and polylactic acid; and a ligand conjugate, as disclosed in US 20140235706 to Zale et al. (BIND Therapeutics), the contents of which are incorporated herein by reference in their entirety. Any of the particle compositions in U.S. Pat. Nos. 8,603,501, 8,603,500, 8,603,499, 8,273,363, 8,246,968, 20130172406 to Zale et al., may also be used.

In some embodiments, the particles comprise a targeting moiety. As a non-limiting example, the particles may comprise about 1 to about 20 mole percent PLA-PEG-basement vascular membrane targeting peptide, wherein the targeting peptide comprises PLA having a number average molecular weight of about 15 to about 20 kDa and PEG having a number average molecular weight of about 4 to about 6 kDa; about 10 to about 25 weight percent anti-neointimal hyperplasia (NIH) agent; and about 50 to about 90 weight percent non-targeted polylactic acid-PEG, wherein the therapeutic particle is capable of releasing the anti-NIH agent to a basement vascular membrane of a blood vessel for at least about 8 hours when the therapeutic particle is placed in the blood vessel as disclosed in U.S. Pat. No. 8,563,041 to Grayson et al. (BIND Therapeutics), the contents of which are incorporated herein by reference in their entirety.

In some embodiments, the particles comprise about 40 to about 99% by weight of poly(D,L-lactic)acid-poly(ethylene)glycol copolymer; and about 0.2 to about 10 mole percent PLA-PEG-ligand; wherein the pharmaceutical aqueous suspension have a glass transition temperature between about 39 and 41° C., as disclosed in U.S. Pat. No. 8,518,963 to Ali et al. (BIND Therapeutics), the contents of which are incorporated herein by reference in their entirety.

In some embodiments, the particles comprise about 0.2 to about 35 weight percent of an active agent; about 10 to about 99 weight percent of a diblock poly(lactic) acid-poly(ethylene)glycol copolymer or a diblock poly(lactic)-co-poly (glycolic) acid-poly(ethylene)glycol copolymer; and about 0 to about 75 weight percent poly(lactic) acid or poly(lactic) acid-co-poly (glycolic) acid as disclosed in WO2012166923 to Zale et al. (BIND Therapeutics), the contents of which are incorporated herein by reference in their entirety.

In some embodiments, the particles are long circulating and may be formulated in a biocompatible and injectable formulation. For example, the particles may be a sterile, biocompatible and injectable nanoparticle composition comprising a plurality of long circulating nanoparticles having a diameter of about 70 to about 130 nm, each of the plurality of the long circulating nanoparticles comprising about 70 to about 90 weight percent poly(lactic) acid-co-poly(ethylene) glycol, wherein the weight ratio of poly(lactic) acid to poly(ethylene) glycol is about 15 kDa/2 kDa to about 20 kDa/10 kDa, and a therapeutic agent encapsulated in the nanoparticles as disclosed in US 20140093579 to Zale et al. (BIND Therapeutics), the content of which is incorporated herein by reference in its entirety.

In some embodiments, provided is a reconstituted lyophilized pharmaceutical composition suitable for parenteral administration comprising the particles of the present invention. For example, the reconstituted lyophilized pharmaceutical composition may comprise a 10-100 mg/mL concentration of polymeric nanoparticles in an aqueous medium; wherein the polymeric nanoparticles comprise: a poly(lactic) acid-block-poly(ethylene)glycol copolymer or poly(lactic)-co-poly(glycolic) acid-block-poly(ethylene)glycol copolymer, and a taxane agent; 4 to 6 weight percent sucrose or trehalose; and 7 to 12 weight percent hydroxypropyl β-cyclodextrin, as disclosed in U.S. Pat. No. 8,637,083 to Troiano et al. (BIND Therapeutics), the contents of which are incorporated herein by reference in their entirety. Any pharmaceutical composition in U.S. Pat. Nos. 8,603,535, 8,357,401, 20130230568, 20130243863 to Troiano et al. may also be used.

In some embodiments, the conjugates and/or particles of the invention may be delivered with a bacteriophage. For example, a bacteriophage may be conjugated through a labile/non labile linker or directly to at least 1,000 therapeutic drug molecules such that the drug molecules are conjugated to the outer surface of the bacteriophage as disclosed in US 20110286971 to Yacoby et al., the content of which is incorporated herein by reference in its entirety. According to Yacoby et al., the bacteriophage may comprise an exogenous targeting moiety that binds a cell surface molecule on a target cell.

In some embodiments, the conjugates and/or particles of the invention may be delivered with a dendrimer. The conjugates may be encapsulated in a dendrimer, or disposed on the surface of a dendrimer. For example, the conjugates may bind to a scaffold for dendritic encapsulation, wherein the scaffold is covalently or non-covalently attached to a polysaccharide, as disclosed in US 20090036553 to Piccariello et al., the content of which is incorporated herein by reference in its entirety. The scaffold may be any peptide or oligonucleotide scaffold disclosed by Piccariello et al.

In some embodiments, the conjugates and/or particles of the invention may be delivered by a cyclodextrin. In one embodiment, the conjugates may be formulated with a polymer comprising a cyclodextrin moiety and a linker moiety as disclosed in US 20130288986 to Davis et al., the content of which is incorporated herein by reference in its entirety. Davis et al. also teaches that the conjugate may be covalently attached to a polymer through a tether, wherein the tether comprises a self-cyclizing moiety.

In some embodiments, the conjugates and/or particles of the invention may be delivered with an aliphatic polymer. For example, the aliphatic polymer may comprise polyesters with grafted zwitterions, such as polyester-graft-phosphorylcholine polymers prepared by ring-opening polymerization and click chemistry as disclosed in U.S. Pat. No. 8,802,738 to Emrick; the content of which is incorporated herein by reference in its entirety.

### A. Excipients

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, ( )ey′ alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN® 20], polyoxyethylene sorbitan [TWEENn® 60], polyoxyethylene sorbitan monooleate [TWEEN® 80], sorbitan monopalmitate [SPAN® 40], sorbitan monostearate [SPAN® 60], sorbitan tristearate [SPAN® 65], glyceryl monooleate, sorbitan monooleate [SPAN® 80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC® F 68, POLOXAMER® 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™ KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### B. Lipidoids

Lipidoids may be used to deliver conjugates and nanoparticles of the present invention. Complexes, micelles, liposomes or particles can be prepared containing these lipidoids and therefore, can result in an effective delivery of the conjugates of the present invention, for a variety of therapeutic indications including vaccine adjuvants, following the injection of a lipidoid formulation via localized and/or systemic routes of administration. Lipidoid complexes of conjugates of the present invention can be administered by various means including, but not limited to, intravenous, intramuscular, or subcutaneous routes.

The lipidoid formulations can include particles comprising either 3 or 4 or more components in addition to conjugates and nanoparticles of the present invention.

The use of lipidoid formulations for the localized delivery of conjugates to cells (such as, but not limited to, adipose cells and muscle cells) via either subcutaneous or intramuscular delivery, may not require all of the formulation components desired for systemic delivery, and as such may comprise only the lipidoid and the conjugates.

### C. Liposomes, Lipid Nanoparticles and Lipoplexes

The conjugates and nanoparticles comprising compoments of a CRISPR-Cas system can be formulated using one or more liposomes, lipoplexes, or lipid nanoparticles. In one embodiment, pharmaceutical compositions of the conjugates of the invention include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vehicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety).

In one embodiment, the conjugates and nanoparticles comprising compoments of a CRISPR-Cas system may be formulated in a lipid vesicle which may have crosslinks between functionalized lipid bilayers.

In one embodiment, the conjugates and nanoparticles comprising compoments of a CRISPR-Cas system may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326; herein incorporated by reference in its entirety. In another embodiment, the conjugates of the invention may be formulated in a lipid-polycation complex which may further include a neutral lipid such as, but not limited to, cholesterol or dioleoyl phosphatidylethanolamine (DOPE).

The liposome formulation may be influenced by, but not limited to, the selection of the cationic lipid component, the degree of cationic lipid saturation, the nature of the PEGylation, ratio of all components and biophysical parameters such as size.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain 1-5% of the lipid molar ratio of PEG-c-DOMG as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol) or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In one embodiment, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302, 7,404,969 and 8,283,333 and US Patent Publication No. US20100036115 and US20120202871; each of which is herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365 and WO2012044638; each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115; the contents of each of which are herein incorporated by reference in their entirety.

In one embodiment, the cationic lipid may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724 and WO201021865; each of which is herein incorporated by reference in their entirety.

In one embodiment, the LNP formulation may be formulated by the methods described in International Publication Nos. WO2011127255 or WO2008103276, each of which is herein incorporated by reference in their entirety. As a non-limiting example, conjugates described herein may be encapsulated in LNP formulations as described in WO2011127255 and/or WO2008103276; each of which is herein incorporated by reference in their entirety. As another non-limiting example, conjugates described herein may be formulated in a nanoparticle to be delivered by a parenteral route as described in U.S. Pub. No. 20120207845; herein incorporated by reference in its entirety.

The nanoparticle formulations may be a carbohydrate nanoparticle comprising a carbohydrate carrier and a conjugate. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; herein incorporated by reference in its entirety).

Nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosa tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photo bleaching (FRAP) and high resolution multiple particle tracking (MPT). As a non-limiting example, compositions which can penetrate a mucosal barrier may be made as described in U.S. Pat. No. 8,241,670, herein incorporated by reference in its entirety.

Nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. The polymeric material may additionally be irradiated. As a non-limiting example, the polymeric material may be gamma irradiated (See e.g., International App. No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), and trimethylene carbonate, polyvinylpyrrolidone. The lipid nanoparticle may be coated or associated with a co-polymer such as, but not limited to, a block co-polymer, and (poly(ethylene glycol))-(poly(propylene oxide))-(poly(ethylene glycol)) triblock copolymer (see e.g., US Publication 20120121718 and US Publication 20100003337 and U.S. Pat. No. 8,263,665; each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; herein incorporated by reference in its entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

In one embodiment, the conjugate and nanoparticles comprising compoments of a CRISPR-Cas system is formulated as a lipoplex, such as, without limitation, the ATUPLEX™ system, the DACC system, the DBTC system and other conjugate-lipoplex technology from Silence Therapeutics (London, United Kingdom), STEMFECT™ from STEMGENT® (Cambridge, Mass.), and polyethylenimine (PEI) or protamine-based targeted and non-targeted delivery of therapeutic agents (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo, Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In one embodiment such formulations may also be constructed or compositions altered such that they passively or actively are directed to different cell types in vivo, including but not limited to cell lines, primary cells (e.g., immune cells, neural cells and endothelial cells) and tumor cells. Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011, 12:2708-2714; Zhao et al., Expert Opin Drug Deliv. 2008, 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer, J Control Release. 2010, 20:63-68; Peer et al., Proc Natl Acad Sci USA. 2007, 104:4095-4100; Kim et al., Methods Mol Biol. 2011, 721:339-353; Subramanya et al., Mol Ther. 2010, 18:2028-2037; Song et al., Nat Biotechnol. 2005, 23:709-717; Peer et al., Science. 2008, 319:627-630; Peer and Lieberman, Gene Ther. 2011, 18:1127-1133; all of which are incorporated herein by reference in its entirety).

In one embodiment, the conjugates and nanoparticles comprising compoments of a CRISPR-Cas system are formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702; herein incorporated by reference in its entirety).

In one embodiment, the conjugates and nanoparticles comprising compoments of a CRISPR-Cas system can be formulated for controlled release and/or targeted delivery. As used herein, “controlled release” refers to a pharmaceutical composition or compound release profile that conforms to a particular pattern of release to effect a therapeutic outcome. In one embodiment, the conjugates of the invention may be encapsulated into a delivery agent described herein and/or known in the art for controlled release and/or targeted delivery. As used herein, the term “encapsulate” means to enclose, surround or encase. As it relates to the formulation of the conjugates of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of conjugate of the invention may be enclosed, surrounded or encased within the particle. “Partially encapsulation” means that less than 10, 10, 20, 30, 40 50 or less of the conjugate of the invention may be enclosed, surrounded or encased within the particle. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the particle.

In another embodiment, the conjugates of the invention may be encapsulated into a nanoparticle or a rapidly eliminated nanoparticle and the nanoparticles or a rapidly eliminated nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

In another embodiment, the nanoparticle may be encapsulated into any polymer known in the art which may form a gel when injected into a subject. As a non-limiting example, the nanoparticle may be encapsulated into a polymer matrix which may be biodegradable.

In one embodiment, the conjugate formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the controlled release and/or targeted delivery formulation may comprise at least one degradable polyester which may contain polycationic side chains. Degradable polyesters include, but are not limited to, poly (serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the conjugate and nanoparticles of the present invention comprising compoments of a CRISPR-Cas system of the present invention may be encapsulated in a therapeutic nanoparticle. Therapeutic nanoparticles may be formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286 and US20120288541, and U.S. Pat. Nos. 8,206,747, 8,293,276 8,318,208 and 8,318,211; each of which is herein incorporated by reference in their entirety. In another embodiment, therapeutic polymer nanoparticles may be identified by the methods described in US Pub No. US20120140790, herein incorporated by reference in its entirety.

In one embodiment, the present nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the conjugate of the present invention (see International Pub No. 2010075072 and US Pub No. US20100216804, US20110217377 and US20120201859, each of which is herein incorporated by reference in their entirety).

In one embodiment, the present nanoparticles may be formulated to be target specific. As a non-limiting example, the therapeutic nanoparticles may include a corticosteroid (see International Pub. No. WO2011084518 herein incorporated by reference in its entirety). As a non-limiting example, the therapeutic nanoparticles may be formulated in nanoparticles described in International Pub No. WO2008121949, WO2010005726, WO2010005725, WO2011084521 and US Pub No. US20100069426, US20120004293 and US20100104655, each of which is herein incorporated by reference in their entirety.

In one embodiment, the nanoparticles of the present invention comprising compoments of a CRISPR-Cas system may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In one embodiment, the present nanoparticle comprises a diblock copolymer. In one embodiment, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

As a non-limiting example, the nanoparticle comprises a PLGA-PEG block copolymer (see US Pub. No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968, herein incorporated by reference in its entirety).

In one embodiment, the therapeutic nanoparticle may comprise a multiblock copolymer (See e.g., U.S. Pat. Nos. 8,263,665 and 8,287,910; each of which is herein incorporated by reference in its entirety).

In one embodiment, the block copolymers described herein may be included in a polyion complex comprising a non-polymeric micelle and the block copolymer. (See e.g., U.S. Pub. No. 20120076836; herein incorporated by reference in its entirety).

In one embodiment, the present nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the present nanoparticles may comprise at least one cationic polymer described herein and/or known in the art.

In one embodiment, the present nanoparticles may comprise at least one amine-containing polymer such as, but not limited to polylysine, polyethylene imine, poly(amidoamine) dendrimers, poly(beta-amino esters) (See e.g., U.S. Pat. No. 8,287,849; herein incorporated by reference in its entirety) and combinations thereof.

In one embodiment, the therapeutic nanoparticles may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In another embodiment, the present nanoparticle may include a conjugation of at least one targeting ligand. The targeting ligand may be any ligand known in the art such as, but not limited to, a monoclonal antibody. (Kirpotin et al, Cancer Res. 2006 66:6732-6740; herein incorporated by reference in its entirety).

In one embodiment, the present nanoparticle may be formulated in an aqueous solution which may be used to target cancer (see International Pub No. WO2011084513 and US Pub No. US20110294717, each of which is herein incorporated by reference in their entirety).

In one embodiment, the conjugates and nanoparticles of the present invention comprising compoments of a CRISPR-Cas system may be encapsulated in, linked to and/or associated with synthetic nanocarriers. Synthetic nanocarriers include, but are not limited to, those described in International Pub. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411 and WO2012149454 and US Pub. Nos. US20110262491, US20100104645, US20100087337 and US20120244222, each of which is herein incorporated by reference in their entirety. The synthetic nanocarriers may be formulated using methods known in the art and/or described herein. As a non-limiting example, the synthetic nanocarriers may be formulated by the methods described in International Pub Nos. WO2010005740, WO2010030763 and WO201213501 and US Pub. Nos. US20110262491, US20100104645, US20100087337 and US20120244222, each of which is herein incorporated by reference in their entirety. In another embodiment, the synthetic nanocarrier formulations may be lyophilized by methods described in International Pub. No. WO2011072218 and U.S. Pat. No. 8,211,473; each of which is herein incorporated by reference in their entirety.

In one embodiment, the synthetic nanocarriers may contain reactive groups to release the conjugates described herein (see International Pub. No. WO20120952552 and US Pub No. US20120171229, each of which is herein incorporated by reference in their entirety).

In one embodiment, the synthetic nanocarriers may be formulated for targeted release. In one embodiment, the synthetic nanocarrier is formulated to release the conjugates at a specified pH and/or after a desired time interval. As a non-limiting example, the synthetic nanoparticle may be formulated to release the conjugates after 24 hours and/or at a pH of 4.5 (see International Pub. Nos. WO2010138193 and WO2010138194 and US Pub Nos. US20110020388 and US20110027217, each of which is herein incorporated by reference in their entirety).

In one embodiment, the synthetic nanocarriers may be formulated for controlled and/or sustained release of conjugates described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In one embodiment, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Pub. No. 20120282343; herein incorporated by reference in its entirety.

### D. Polymers, Biodegradable Nanoparticles, and Core-Shell Nanoparticles

The conjugates and nanoparticles of the invention comprising compoments of a CRISPR-Cas system can be formulated using natural and/or synthetic polymers. Non-limiting examples of polymers which may be used for delivery include, but are not limited to, DYNAMIC POLYCONJUGATE® (Arrowhead Research Corp., Pasadena, Calif.) formulations from MIRUS® Bio (Madison, Wis.) and Roche Madison (Madison, Wis.), PHASERX™ polymer formulations such as, without limitation, SMARTT POLYMER TECHNOLOGY™ (Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers, RONDEL™ (RNAi/Oligonucleotide Nanoparticle Delivery) polymers (Arrowhead Research Corporation, Pasadena, Calif.) and pH responsive co-block polymers such as, but not limited to, PHASERX™ (Seattle, Wash.).

A non-limiting example of chitosan formulation includes a core of positively charged chitosan and an outer portion of negatively charged substrate (U.S. Pub. No. 20120258176; herein incorporated by reference in its entirety). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

In one embodiment, the polymers used in the present invention have undergone processing to reduce and/or inhibit the attachment of unwanted substances such as, but not limited to, bacteria, to the surface of the polymer. The polymer may be processed by methods known and/or described in the art and/or described in International Pub. No. WO2012150467, herein incorporated by reference in its entirety.

A non-limiting example of PLGA formulations include, but are not limited to, PLGA injectable depots (e.g., ELIGARD® which is formed by dissolving PLGA in 66% N-methyl-2-pyrrolidone (NMP) and the remainder being aqueous solvent and leuprolide. Once injected, the PLGA and leuprolide peptide precipitates into the subcutaneous space).

In one embodiment, the compositions may be sustained release formulations. In a further embodiment, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

As a non-limiting example, conjugates and nanoparticles of the present invention comprising compoments of a CRISPR-Cas system may be formulated in PLGA microspheres by preparing the PLGA microspheres with tunable release rates (e.g., days and weeks) and encapsulating the conjugate in the PLGA microspheres while maintaining the integrity of the conjugate during the encapsulation process. EVAc are non-biodegradable, biocompatible polymers which are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C. PEG-based surgical sealants comprise two synthetic PEG components mixed in a delivery device which can be prepared in one minute, seals in 3 minutes and is reabsorbed within 30 days. GELSITE® and natural polymers are capable of in-situ gelation at the site of administration. They have been shown to interact with protein and peptide therapeutic candidates through ionic interaction to provide a stabilizing effect.

Polymer formulations can also be selectively targeted through expression of different ligands as exemplified by, but not limited by, folate, transferrin, and N-acetylgalactosamine (GalNAc) (Benoit et al., Biomacromolecules. 2011 12:2708-2714; Rozema et al., Proc Natl Acad Sci USA. 2007 104:12982-12887; Davis, Mol Pharm. 2009, 6:659-668; Davis, Nature, 2010, 464:1067-1070; each of which is herein incorporated by reference in its entirety).

The conjugates and nanoparticles of the invention comprising components of a CRISPR-Cas system may be formulated with or in a polymeric compound. The polymer may include at least one polymer such as, but not limited to, polyethenes, polyethylene glycol (PEG), poly(l-lysine)(PLL), PEG grafted to PLL, cationic lipopolymer, biodegradable cationic lipopolymer, polyethylenimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly [α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

As a non-limiting example, the conjugate and nanoparticle of the invention comprising compoments of a CRISPR-Cas system may be formulated with the polymeric compound of PEG grafted with PLL as described in U.S. Pat. No. 6,177,274; herein incorporated by reference in its entirety. In another example, the conjugate may be suspended in a solution or medium with a cationic polymer, in a dry pharmaceutical composition or in a solution that is capable of being dried as described in U.S. Pub. Nos. 20090042829 and 20090042825; each of which are herein incorporated by reference in their entireties.

As another non-limiting example the conjugate and nanoparticle of the invention comprising components of a CRISPR-Cas system may be formulated with a PLGA-PEG block copolymer (see US Pub. No. US20120004293 and U.S. Pat. No. 8,236,330, each of which are herein incorporated by reference in their entireties) or PLGA-PEG-PLGA block copolymers (See U.S. Pat. No. 6,004,573, herein incorporated by reference in its entirety). As a non-limiting example, the conjugate of the invention may be formulated with a diblock copolymer of PEG and PLA or PEG and PLGA (see U.S. Pat. No. 8,246,968, herein incorporated by reference in its entirety).

A polyamine derivative may be used to deliver conjugates and nanoparticles of the invention or to treat and/or prevent a disease or to be included in an implantable or injectable device (U.S. Pub. No. 20100260817 herein incorporated by reference in its entirety). As a non-limiting example, a pharmaceutical composition may include the conjugate and nanoparticle of the invention comprising components of a CRISPR-Cas system and the polyamine derivative described in U.S. Pub. No. 20100260817 (the contents of which are incorporated herein by reference in its entirety). As a non-limiting example the conjugate and nanoparticle of the invention comprising components of a CRISPR-Cas system may be delivered using a polyamide polymer such as, but not limited to, a polymer comprising a 1,3-dipolar addition polymer prepared by combining a carbohydrate diazide monomer with a dilkyne unite comprising oligoamines (U.S. Pat. No. 8,236,280; herein incorporated by reference in its entirety).

The conjugate and nanoparticle of the invention comprising components of a CRISPR-Cas system may be formulated with at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the conjugate and nanoparticle of the invention comprising components of a CRISPR-Cas system may be formulated with at least one polymer and/or derivatives thereof described in International Publication Nos. WO2011115862, WO2012082574 and WO2012068187 and U.S. Pub. No. 20120283427, each of which are herein incorporated by reference in their entireties. In another embodiment, the conjugates of the invention may be formulated with a polymer of formula Z as described in WO2011115862, herein incorporated by reference in its entirety. In yet another embodiment, the conjugates of the invention may be formulated with a polymer of formula Z, Z′ or Z″ as described in International Pub. Nos. WO2012082574 or WO2012068187, each of which are herein incorporated by reference in their entireties. The polymers formulated with the conjugates of the present invention may be synthesized by the methods described in International Pub. Nos. WO2012082574 or WO2012068187, each of which are herein incorporated by reference in their entireties.

Formulations of conjugates and nanoparticles of the invention may include at least one amine-containing polymer such as, but not limited to polylysine, polyethylene imine, poly(amidoamine) dendrimers or combinations thereof.

For example, the conjugate and nanoparticle of the invention comprising components of a CRISPR-Cas system may be formulated in a pharmaceutical compound including a poly(alkylene imine), a biodegradable cationic lipopolymer, a biodegradable block copolymer, a biodegradable polymer, or a biodegradable random copolymer, a biodegradable polyester block copolymer, a biodegradable polyester polymer, a biodegradable polyester random copolymer, a linear biodegradable copolymer, PAGA, a biodegradable cross-linked cationic multi-block copolymer or combinations thereof. The biodegradable cationic lipopolymer may be made by methods known in the art and/or described in U.S. Pat. No. 6,696,038, U.S. App. Nos. 20030073619 and 20040142474 each of which is herein incorporated by reference in their entireties. The poly(alkylene imine) may be made using methods known in the art and/or as described in U.S. Pub. No. 20100004315, herein incorporated by reference in its entirety. The biodegradable polymer, biodegradable block copolymer, the biodegradable random copolymer, biodegradable polyester block copolymer, biodegradable polyester polymer, or biodegradable polyester random copolymer may be made using methods known in the art and/or as described in U.S. Pat. Nos. 6,517,869 and 6,267,987, the contents of which are each incorporated herein by reference in their entirety. The linear biodegradable copolymer may be made using methods known in the art and/or as described in U.S. Pat. No. 6,652,886. The PAGA polymer may be made using methods known in the art and/or as described in U.S. Pat. No. 6,217,912 herein incorporated by reference in its entirety. The PAGA polymer may be copolymerized to form a copolymer or block copolymer with polymers such as but not limited to, poly-L-lysine, polyarginine, polyornithine, histones, avidin, protamines, polylactides and poly(lactide-co-glycolides). The biodegradable cross-linked cationic multi-block copolymers may be made my methods known in the art and/or as described in U.S. Pat. No. 8,057,821 or U.S. Pub. No. 2012009145 each of which are herein incorporated by reference in their entireties. For example, the multi-block copolymers may be synthesized using linear polyethylenimine (LPEI) blocks which have distinct patterns as compared to branched polyethyleneimines. Further, the composition or pharmaceutical composition may be made by the methods known in the art, described herein, or as described in U.S. Pub. No. 20100004315 or U.S. Pat. Nos. 6,267,987 and 6,217,912 each of which are herein incorporated by reference in their entireties.

The conjugate and nanoparticle of the invention comprising components of a CRISPR-Cas system may be formulated with at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

The conjugate and nanoparticle of the invention comprising components of a CRISPR-Cas system may be formulated with at least one cross linkable polyester. Cross linkable polyesters include those known in the art and described in US Pub. No. 20120269761, herein incorporated by reference in its entirety.

In one embodiment, the polymers described herein may be conjugated to a lipid-terminating PEG. As a non-limiting example, PLGA may be conjugated to a lipid-terminating PEG forming PLGA-DSPE-PEG. As another non-limiting example, PEG conjugates for use with the present invention are described in International Publication No. WO2008103276, herein incorporated by reference in its entirety. The polymers may be conjugated using a ligand conjugate such as, but not limited to, the conjugates described in U.S. Pat. No. 8,273,363, herein incorporated by reference in its entirety.

In one embodiment, the conjugate and nanoparticle of the invention comprising components of a CRISPR-Cas system may be conjugated with another compound. Non-limiting examples of conjugates are described in U.S. Pat. Nos. 7,964,578 and 7,833,992, each of which are herein incorporated by reference in their entireties. In another embodiment, the conjugates of the invention may be conjugated with conjugates of formula 1-122 as described in U.S. Pat. Nos. 7,964,578 and 7,833,992, each of which are herein incorporated by reference in their entireties. The modified RNA described herein may be conjugated with a metal such as, but not limited to, gold. (See e.g., Giljohann et al. Journ. Amer. Chem. Soc. 2009 131(6): 2072-2073; herein incorporated by reference in its entirety). In another embodiment, the conjugates of the invention may be conjugated and/or encapsulated in gold-nanoparticles. (Interantional Pub. No. WO201216269 and U.S. Pub. No. 20120302940; each of which is herein incorporated by reference in its entirety).

In one embodiment, the polymer formulation of the present invention may be stabilized by contacting the polymer formulation, which may include a cationic carrier, with a cationic lipopolymer which may be covalently linked to cholesterol and polyethylene glycol groups. The polymer formulation may be contacted with a cationic lipopolymer using the methods described in U.S. Pub. No. 20090042829 herein incorporated by reference in its entirety. The cationic carrier may include, but is not limited to, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B—[N—(N′,N′-Dimethylaminoethane)-carbamoyl]Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof.

The conjugate and nanoparticle of the invention comprising components of a CRISPR-Cas system may be formulated in a polyplex of one or more polymers (U.S. Pub. No. 20120237565 and 20120270927; each of which is herein incorporated by reference in its entirety). In one embodiment, the polyplex comprises two or more cationic polymers. The catioinic polymer may comprise a poly (ethylene imine) (PEI) such as linear PEI.

The conjugate and nanoparticle of the invention comprising components of a CRISPR-Cas system can also be formulated as a nanoparticle using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so that delivery of the conjugates of the invention may be enhanced (Wang et al., Nat Mater. 2006, 5:791-796; Fuller et al., Biomaterials. 2008, 29:1526-1532; DeKoker et al., Adv Drug Deliv Rev. 2011, 63:748-761; Endres et al., Biomaterials. 2011, 32:7721-7731; Su et al., Mol Pharm. 2011 Jun. 6; 8(3):774-87; each of which is herein incorporated by reference in its entirety). As a non-limiting example, the nanoparticle may comprise a plurality of polymers such as, but not limited to hydrophilic-hydrophobic polymers (e.g., PEG-PLGA), hydrophobic polymers (e.g., PEG) and/or hydrophilic polymers (International Pub. No. WO20120225129; herein incorporated by reference in its entirety).

Biodegradable calcium phosphate nanoparticles in combination with lipids and/or polymers have been shown to deliver therapeutic agents in vivo. In one embodiment, a lipid coated calcium phosphate nanoparticle, which may also contain a targeting ligand such as anisamide, may be used to deliver the conjugate of the present invention. For example, to effectively deliver a therapeutic agent in a mouse metastatic lung model a lipid coated calcium phosphate nanoparticle was used (Li et al., J Contr Rel. 2010, 142: 416-421; Li et al., J Contr Rel. 2012, 158:108-114; Yang et al., Mol Ther. 2012, 20:609-615; which is herein incorporated by reference in its entirety). This delivery system combines both a targeted nanoparticle and a component to enhance the endosomal escape, calcium phosphate, in order to improve delivery of the therapeutic agent.

In one embodiment, a PEG-charge-conversional polymer (Pitella et al., Biomaterials. 2011, 32:3106-3114) may be used to form a nanoparticle to deliver the conjugate of the present invention. The PEG-charge-conversional polymer may improve upon the PEG-polyanion block copolymers by being cleaved into a polycation at acidic pH, thus enhancing endosomal escape.

The use of core-shell nanoparticles has additionally focused on a high-throughput approach to synthesize cationic cross-linked nanogel cores and various shells (Siegwart et al., Proc Natl Acad Sci USA. 2011, 108:12996-13001). The complexation, delivery, and internalization of the polymeric nanoparticles can be precisely controlled by altering the chemical composition in both the core and shell components of the nanoparticle. For example, the core-shell nanoparticles may efficiently deliver a therapeutic agent to mouse hepatocytes after they covalently attach cholesterol to the nanoparticle.

The use of core-shell nanoparticles has additionally focused on a high-throughput approach to synthesize cationic cross-linked nanogel cores and various shells (Siegwart et al., Proc Natl Acad Sci USA. 2011, 108:12996-13001). The complexation, delivery, and internalization of the polymeric nanoparticles can be precisely controlled by altering the chemical composition in both the core and shell components of the nanoparticle. For example, the core-shell nanoparticles may efficiently deliver a therapeutic agent to mouse hepatocytes after they covalently attach cholesterol to the nanoparticle.

In one embodiment, the lipid nanoparticles may comprise a core of the conjugates disclosed herein and a polymer shell. The polymer shell may be any of the polymers described herein and are known in the art. In an additional embodiment, the polymer shell may be used to protect the modified nucleic acids in the core.

Core-shell nanoparticles for use with the conjugates of the present invention are described and may be formed by the methods described in U.S. Pat. No. 8,313,777 herein incorporated by reference in its entirety.

### E. Inorganic Nanoparticles

Inorganic nanoparticles exhibit a combination of physical, chemical, optical and electronic properties and provide a highly multifunctional platform to image and diagnose diseases, to selectively deliver therapeutic agents, and to sensitive cells and tissues to treatment regiments. Not wishing to be bound to any theory, enhanced permeability and retention (EPR) effect provides a basis for the selective accumulation of many high-molecular-weight drugs. Circulating inorganic nanoparticles preferentially accumulate at tumor sites and in inflamed tissues (Yuan et al., Cancer Res., vol. 55(17):3752-6, 1995, the contents of which are incorporated herein by reference in their entirety) and remain lodged due to their low diffusivity (Pluen et al., PNAS, vol. 98(8):4628-4633, 2001, the contents of which are incorporated herein by reference in their entirety). The size of the inorganic nanoparticles may be 10 nm-500 nm, 10 nm-100 nm or 100 nm-500 nm. The inorganic nanoparticles may comprise metal (gold, iron, silver, copper, nickel, etc.), oxides (ZnO, TiO₂, Al₂O₃, SiO₂, iron oxide, copper oxide, nickel oxide, etc.), or semiconductor (CdS, CdSe, etc.). The inorganic nanoparticles may also be perfluorocarbon or FeCo.

Inorganic nanoparticles have high surface area per unit volume. Therefore, they may be loaded with therapeutic drugs and imaging agents at high densitives. A variety of methods may be used to load therapeutic drugs into/onto the inorganic nanoparticles, including but not limited to, colvalent bonds, electrostatic interactions, entrapment, and encapsulation. In addition to therapeutic agent drug loads, the inorganic nanoparticles may be funcationalized with targeting moieties, such as tumor-targeting ligands, on the surface. Formulating therapeutic agents with inorganic nanoparticles allows imaging, detection and monitoring of the therapeutic agents.

In one embodiment, the conjugate and nanoparticle of the invention comprising components of a CRISPR-Cas system is hydrophobic and may be form a kinetically stable complex with gold nanoparticles funcationalized with water-soluble zwitterionic ligands disclosed by Kim et al. (Kim et al., JACS, vol. 131(4):1360-1361, 2009, the contents of which are incorporated herein by reference in their entirety). Kim et al. demonstrated that hydrophobic drugs carried by the gold nanoparticles are efficiently released into cells with little or no cellular uptake of the gold nanoparticles.

In one embodiment, the conjugate and nanoparticle of the invention comprising components of a CRISPR-Cas system may be formulated with gold nanoshells. As a non-limiting example, the conjugates may be delivered with a temperature sensitive system comprising polymers and gold nanoshells and may be released photothermally. Sershen et al. designed a delivery vehicle comprising hydrogel and gold nanoshells, wherein the hydrogels are made of copolymers of N-isopropylacrylamide (NIPAAm) and acrylamide (AAm) and the gold nanoshells are made of gold and gold sulfide (Sershen et al., J Blamed Mater, vol. 51:293-8, 2000, the contents of which are incorporated herein by reference in their entirety). Irradiation at 1064 nm was absorbed by the nanoshells and converted to heat, which led to the collapse of the hydrogen and release of the drug. The conjugate of the invention may also be encapsulated inside hollow gold nanoshells.

In some embodiments, the conjugate and nanoparticle of the invention comprising components of a CRISPR-Cas system may be attached to gold nanoparticles via covalent bonds. Covalent attachment to gold nanoparticles may be achieved through a linker, such as a free thiol, amine or carboxylate functional group. In some embodiments, the linkers are located on the surface of the gold nanoparticles. In some embodiments, the conjugates of the invention may be modified to comprise the linkers. The linkers may comprise a PEG or oligoethylene glycol moiety with varying length to increase the particles' stability in biological environment and to control the density of the drug loads. PEG or oligoethylene glycol moieties also minimize nonspecific adsorption of undesired biomolecules. PEG or oligoethylene gycol moieties may be branched or linear. Tong et al. disclosed that branched PEG moieties on the surface of gold nanoparticles increase circulatory half-life of the gold nanoparticles and reduced serum protein binding (Tong et al., Langmuir, vol. 25(21):12454-9, 2009, the contents of which are incorporated herein by reference in their entirety).

In one embodiment, the conjugate and nanoparticle of the invention comprising components of a CRISPR-Cas system may comprise PEG-thiol groups and may attach to gold nanoparticles via the thiol group. The synthesis of thiol-PEGylated conjugates and the attachment to gold nanoparticles may follow the method disclosed by El-Sayed et al. (El-Sayed et al., Bioconjug. Chem., vol. 20(12):2247-2253, 2010, the contents of which are incorporated herein by reference in their entirety).

In another embodiment, the conjugate and nanoparticle of the invention comprising components of a CRISPR-Cas system may be tethered to an amine-functionalized gold nanoparticles. Lippard et al. disclosed that Pt(IV) prodrugs may be delivered with amine-functionalized polyvalent oligonucleotide gold nanoparticles and are only activated into their active Pt(II) forms after crossing the cell membrane and undergoing intracellular reduction (Lippard et al., JACS, vol. 131(41):14652-14653, 2009, the contents of which are incorporated herein by reference in their entirety). The cytotoxic effects for the Pt(IV)-gold nanoparticle complex are higher than the free Pt(IV) drugs and free cisplatin.

In some embodiments, the conjugate and nanoparticle of the invention comprising components of a CRISPR-Cas system may be formulated with magnetic nanoparticle such as iron, cobalt, nickel and oxides thereof, or iron hydroxide nanoparticles. Localized magnetic field gradients may be used to attract magnetic nanoparticles to a chosen site, to hold them until the therapy is complete, and then to remove them. Magnetic nanoparticles may also be heated by magnetic fields. Alexiou et al. prepared an injection of magnetic particle, Ferro fluids (FFs), bound to anticancer agents and then concentrated the particles in the desired tumor area by an external magnetic field (Alexiou et al., Cancer Res. vol. 60(23):6641-6648, 2000, the contents of which are incorporated herein by reference in their entirety). The desorption of the anticancer agent took place within 60 min to make sure that the drug can act freely once localized to the tumor by the magnetic field.

In some embodiments, the conjugates and nanoparticles of the invention comprising components of a CRISPR-Cas system are loaded onto iron oxide nanoparticles. In some embodiments, the conjugates of the invention are formulated with super paramagnetic nanoparticles based on a core consisting of iron oxides (SPION). SPION are coated with inorganic materials (silica, gold, etc.) or organic materials (phospholipids, fatty acids, polysaccharides, peptides or other surfactants and polymers) and can be further functionalized with drugs, proteins or plasmids.

In one embodiment, water-dispersible oleic acid (OA)-poloxamer-coated iron oxide magnetic nanoparticles disclosed by Jain et al. (Jain, Mol. Pharm., vol. 2(3):194-205, 2005, the contents of which are incorporated herein by reference in their entirety) may be used to deliver the conjugates of the invention. Therapeutic drugs partition into the OA shell surrounding the iron oxide nanoparticles and the poloxamer copolymers (i.e., Pluronics) confers aqueous dispersity to the formulation. According to Jain et al., neither the formulation components nor the drug loading affected the magnetic properties of the core iron oxide nanoparticles. Sustained release of the therapeutic drugs was achieved.

In one embodiment, the conjugates and nanoparticles of the invention comprising components of a CRISPR-Cas system are bonded to magnetic nanoparticles with a linker. The linker may be a linker capable of undergoing an intramolecular cyclization to release the conjugates of the invention. Any linker and nanoparticles disclosed in WO2014124329 to Knipp et al., the contents of which are incorporated herein by reference in their entirety, may be used. The cyclization may be induced by heating the magnetic nanoparticle or by application of an alternating electromagnetic field to the magnetic nanoparticle.

In one embodiment, the conjugate and nanoparticle of the invention comprising components of a CRISPR-Cas system may be delivered with a drug delivery system disclosed in U.S. Pat. No. 7,329,638 to Yang et al., the contents of which are incorporated herein by reference in their entirety. The drug delivery system comprises a magnetic nanoparticle associated with a positively charged cationic molecule, at least one therapeutic agent and a molecular recognition element.

In one embodiment, nanoparticles having a phosphate moiety are used to deliver the conjugates of the invention. The phosphate-containing nanoparticle disclosed in U.S. Pat. No. 8,828,975 to Hwu et al., the contents of which are incorporated herein by reference in their entirety, may be used. The nanoparticles may comprise gold, iron oxide, titanium dioxide, zinc oxide, tin dioxide, copper, aluminum, cadmium selenide, silicon dioxide or diamond. The nanoparticles may contain a PEG moiety on the surface.

### E. Peptides and Proteins

The conjugate and nanoparticle of the invention comprising components of a CRISPR-Cas system can be formulated with peptides and/or proteins in order to increase penetration of cells by the conjugates of the invention. In one embodiment, peptides such as, but not limited to, cell penetrating peptides and proteins and peptides that enable intracellular delivery may be used to deliver pharmaceutical formulations. A non-limiting example of a cell penetrating peptide which may be used with the pharmaceutical formulations of the present invention include a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al., Mol. Ther. 3(3):310-8 (2001); Lange′, Cell-Penetrating Peptides: Processes and Applications (CRC Press, Boca Raton Fla., 2002); El-Andaloussi et al., Curr. Pharm. Des. 2003, 11(28):3597-611; and Deshayes et al., Cell. Mol. Life Sci. 2005, 62(16):1839-49, all of which are incorporated herein by reference). The compositions can also be formulated to include a cell penetrating agent, e.g., liposomes, which enhance delivery of the compositions to the intracellular space. The conjugates of the invention may be complexed to peptides and/or proteins such as, but not limited to, peptides and/or proteins from Aileron Therapeutics (Cambridge, Mass.) and Permeon Biologics (Cambridge, Mass.) in order to enable intracellular delivery (Cronican et al., ACS Chem. Biol. 2010, 5:747-752; McNaughton et al., Proc. Natl. Acad. Sci. USA 2009, 106:6111-6116; Sawyer, Chem Biol Drug Des. 2009, 73:3-6; Verdine and Hilinski, Methods Enzymol. 2012, 503:3-33; all of which are herein incorporated by reference in its entirety). In one embodiment, the cell-penetrating polypeptide may comprise a first domain and a second domain. The first domain may comprise a supercharged polypeptide. The second domain may comprise a protein-binding partner. As used herein, “protein-binding partner” includes, but are not limited to, antibodies and functional fragments thereof, scaffold proteins, or peptides. The cell-penetrating polypeptide may further comprise an intracellular binding partner for the protein-binding partner. The cell-penetrating polypeptide may be capable of being secreted from a cell where conjugates of the invention may be introduced.

### IV. Administration, Dose and Dosage Form

Administration:

Compositions and formulations containing an effective amount of conjugates or particles of the present invention may be administered to a subject in need thereof by any route which results in a therapeutically effective outcome in said subject. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them cross the blood-brain barrier, vascular barrier, or other epithelial barrier.

Dose and Dosage Forms:

Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

As used herein, a “split dose” is the division of single unit dose or total daily dose into two or more doses, e.g., two or more administrations of the single unit dose. As used herein, a “single unit dose” is a dose of any therapeutic administed in one dose/at one time/single route/single point of contact, i.e., single administration event. As used herein, a “total daily dose” is an amount given or prescribed in 24 hr. period. It may be administered as a single unit dose.

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, and subcutaneous).

In some embodiments, the dosage forms may be liquid dosage forms. Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMOPHOR®, alcohols, oils, modified oils, glycols, polysorbates, cyclodextrins, polymers, and/or combinations thereof.

In certain embodiments, the dosages forms may be injectable. Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of the compounds then depends upon its rate of dissolution which, in turn, may depend upon crystal size and crystalline form. Injectable depot forms are made by forming microencapsule matrices of the conjugates in biodegradable polymers such as polylactide-polyglycolide. Depending upon the ratio of conjugates to polymer and the nature of the particular polymer employed, the rate of active agents in the conjugates can be controlled. Examples of other biodegradable polymers include, but are not limited to, poly(orthoesters) and poly(anhydrides). Depot injectable formulations may be prepared by entrapping the conjugates in liposomes or microemulsions which are compatible with body tissues.

In some embodiments, solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### V. Application

Conjugates with sgRNAs as active agents, nanoparticles and formulations comprising components of the CRISPR-Cas system, may be applied to edit a nucleic acid sequence in a cell or organism as research tools or therapeutics.

In some embodiments, compositions of the present invention comprising the CRISPR-Cas system may be used as genetic editing technologies. The CRISPR-Cas systems of the present invention can be used to alter any target polynucleotide sequence in any genome. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence. In other embodiments, a target polynucleotide sequence is a pathogenic genomic sequence. Exemplary pathogenic genomic sequences include, but are not limited to a viral genomic sequence, a bacterial genomic sequence, a fungal genomic sequence, or a parasitic genomic sequence.

In some embodiments, compositions of the present invention comprising components of the CRISPR-Cas system may be used to alter a target polynucleotide sequence in a cell for any purpose. In one embodiment, the target polynucleotide sequence in a cell is altered using compositions of the present invention comprising the CRISPR-Cas system to generate a mutate cell, which results in a genotype that differs from its original genotype. In some examples, the target polynucleotide sequence in a cell is altered to correct or repair a genetic mutation (e.g., to restore a normal phenotype to the cell). In other examples, the target polynucleotide sequence in a cell is altered to induce a genetic mutation (e.g., to disrupt the function of a gene or genomic element). In some embodiments, the alteration may be a homozygous alteration or a heterozygous alternation. In some embodiments, the alteration may be an insertion, deletion, or the combination thereof. As will be appreciated by those skilled in the art, an insertion/deletion in a coding region of a genomic sequence will result in a frameshift mutation or a premature stop codon. In some embodiments, the alteration may be a point mutation. As used herein, “point mutation” refers to a substitution that replaces one of the nucleotides in a target polynucleotide.

In some embodiments, compositions of the present invention comprising the CRISPR-Cas system may be used to generate a knock-out of a target polynucleotide sequence. The knocking out of a selected polynucleotide sequence can be useful for many applications, such as knocking out a target polynucleotide sequence in a cell clone in vitro for research purposes; and knocking out a target polynucleotide sequence ex vivo for treating or preventing a disorder associated with increased expression of the target polynucleotide sequence. As used herein, the term “knock out” includes deleting all or a portion of the target polynucleotide sequence in a way that mutes the function of the target polynucleotide sequence.

In some embodiments, the alternation may result in a change of the target polynucleotide sequence from an undesired sequence to a desired sequence. In one example, composition of the present invention comprising the CRISPR-Cas system may be used to correct any type of mutation or error in a target polynucleotide sequence, including but not limited to inserting a nucleotide sequence that is missing from a target polynucleotide sequence due to a deletion, deleting a nucleotide sequence from a target polynucleotide sequence due to an insertion mutation, and replacing an incorrect nucleotide sequence with a correct nucleotide sequence.

In some embodiments, compositions of the present invention may be used to alter gene expression. Catalytically inactive Cas9 (dCas9) guided to the promoter region of a gene can repress transcription by interfering with transcriptional elongation. Transcriptional repression can be enhanced by fusing a transcriptional repression domain to dCas9. Likewise, dCas9 can be fused to a transactivation domain and be used to upregulate the expression of a gene.

In some embodiments, the alternation may result in reduced or increased expression of a target polynucleotide sequence. The terms “decrease,” “reduced,” “reduction,” and “decrease” are all used herein generally to mean a decrease by a statistically significant amount, for example, a decrease by at least 10%, or at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. The terms “increased”, “increase” or “enhance” or “activate” are all used herein to generally mean an increase by a statically significant amount, for example, an increase of at least 10%, or at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

In some embodiments, compositions of the present invention comprising components of a CRISPR-Cas system may be used as therapeutics. In one example, compositions comprising the CRISPR-Cas system may be used to correct disease-causing genetic mutations as remedies to genetic disorders. Compositions comprising the CRISPR-Cas system may be used as a therapeutic against viral infection by inactivating viral gene expression and replication such as HIV viruses. In another example, compositions comprising the CRISPR-Cas system may be used to engineer chimeric antigen receptor T cells (CAR-Ts) for the purpose of developing CRISPR-Cas-based CAR therapies (Mullard, Nat Rev Drug Discov. 2015; 14:82). Other non-limiting examples include correction of mutations in a human cell line that causes cystic fibrosis (Wu et al., Cell Stem Cell. 2013; 13:659-662). Other applications of the CRISPR-Cas system for treatments of disorders are also disclosed in PCT patent publication NOs.: WO2015089419; WO2015077058; WO2014204728; US patent publication NOs.: 20150218253; 20150176013; 20150166969; 20150152436; and 20150071889; the content of each of which is incorporated herein by reference in their entirety.

In accordance with the present invention, the nanoparticles may further comprise a homology-driven repair template along with the components of the CRISPR-Cas system.

In some aspects, compositions of the present invention comprising the CRISPR-Cas system may be used to label a genetic element in a cell or organism; and/or image various elements in the genome of live cells.

Compositions of the present invention comprising the CRISPR-Cas system may also be used to enrich target nucleic acids from a DNA or RNA source, such as enriching a nucleic acid fragment from a genome.

Compositions of the present invention comprising the CRISPR-Cas system can edit the target polynucleotide sequence with higher efficiency and specificity as compared to other CRISPR-Cas systems in the art. In some embodiments, the efficiency of editing the target sequence in a genome may be at least about 10% to at least about 85%. The off-target frequency may be reduced as well.

In some embodiments, the conjugates or particles of the present invention may be combined with at least one other active agent to form a composition. The at least one active agent may be a therapeutic, prophylactic, diagnostic, or nutritional agent. It may be a small molecule, protein, peptide, lipid, glycolipid, glycoprotein, lipoprotein, carbohydrate, sugar, or nucleic acid. The conjugates or particles of the present invention and the at least one other active agent may have the same target and/or treat the same disease.

In some embodiments, the at least one other active agent may be agents to augment EPR effect in patients, e.g. vascular mediators such as NO, CO, bradykinin, VEGF to further enhance EPR effect thus achieving more tumor accumulation of the conjugates or particles (Yin et al., JSM Clin Oncol Res 2(1): 1010 (2014), the contents of which are incorporated herein by reference in their entirety). Any augmentation agent disclosed in section 5 of Maeda et al., Adv. Drug Deliv. Rev. (2012), the contents of which are incorporated herein by reference in their entirety, may be combined with conjugates or particles of the present invention.

In some embodiments, the conjugates or particles of the present invention may be co-delivered with cells. Conjugates or particles of the present invention may preincubate with cells such as Buffy coat cells, stromal cells, or stem cells.

In some embodiments, conjugates or particles of the present invention may be combined in a depot form with a temporal sequence of release of the conjugates or particles. In some cases, the conjugates have different target genes.

### EXAMPLES

### Example 1: sgRNA Synthesis

sgRNA Sequence Design

A sgRNA molecule that guides the CRISPR-Cas complex to a selected target polynucleotide of interest in a cell is designed and the sequence of the sgRNA is determined by selection of target recognition sequences and prediction of off-sites in the genome. The target recognition sequence (20 nucleotides; 20-mer) is based on the methods discussed by Hsu et al., (Hsu et al., Nat. Biotechnol., 2013; 31: 827-832, the content of which is herein incorporated by reference in its entirety). Each 20-mer is upstream of an NGG PAM site and differs by at least three mismatches from any other 20-mer upstream of an NGG or NAG PAM site.

The sgRNA template consists of an in vitro transcription promoter, 18-20nucleotides target recognitions sequence and a sequence of a tracr sequence for the Cas9 protein from S. pyogenes. sgRNAs for each target polynucleotide are generated by the oligonucleotide assembly methods. The target sequence is synthesized by in vitro transcription or by RNA synthesis.

### RNA Synthesis

Where the source of a reagent is not specifically given herein, such reagent may be obtained from any supplier of reagents for molecular biology at a quality/purity standard for application in molecular biology.

All oligonucleotides are synthesized on an AKTAoligopilot synthesizer. Commercially available controlled pore glass solid support (dT-CPG, 500A, Prime Synthesis) and RNA phosphoramidites with standard protecting groups, 5′-O-dimethoxytrityl N6-benzoyl-2′-t-butyldimethylsilyl-adenosine-3′-O—N,N′-diisopropyl-2-cyanoethylphosphoramidite, 5′-O-dimethoxytrityl-N4-acetyl-2′-t-butyldimethylsilyl-cytidine-3′-O—N,N′-diisopropyl-2-cyanoethylphosphoramidite, 5′-O-dimethoxytrityl-N2-isobutryl-2′-t-butyldimethylsilyl-guanosine-3′-O—N,N′-diisopropyl-2-cyanoethylphosphoramidite, and 5′-O-dimethoxytrityl-2′-t-butyldimethylsilyl-uridine-3′-O—N,N′-diisopropyl-2-cyanoethylphosphoramidite (Pierce Nucleic Acids Technologies) were used for the oligonucleotide synthesis. The 2′-F phosphoramidites, 5′-O-dimethoxytrityl-N4-acetyl-2′-fluro-cytidine-3′-O—N,N′-diisopropyl-2-cyanoethyl-phosphoramidite and 5′-O-dimethoxytrityl-2′-fluoro-uridine-3′-O—N,N′-diisopropyl-2-cyanoethyl-phosphoramidite are purchased from (Promega). All phosphoramidites are used at a concentration of 0.2M in acetonitrile (CH3CN) except for guanosine which is used at 0.2M concentration in 10% THF/ANC (v/v). Coupling/recycling time of 16 minutes is used. The activator is 5-ethyl thiotetrazole (0.75M, American International Chemicals); for the PO-oxidation iodine/water/pyridine is used and for the PS-oxidation PADS (2%) in 2,6-lutidine/ACN (1:1 v/v) is used.

3′-ligand conjugated strands are synthesized using solid support containing the corresponding ligand. For example, the introduction of cholesterol unit in the sequence is performed from a hydroxyprolinol-cholesterol phosphoramidite. Cholesterol is tethered to trans-4-hydroxyprolinol via a 6-aminohexanoate linkage to obtain a hydroxyprolinol-cholesterol moiety. 5′-end Cy-3 and Cy-5.5 (fluorophore) labeled iRNAs are synthesized from the corresponding Quasar-570 (Cy-3) phosphoramidite are purchased from Biosearch Technologies. Conjugation of ligands to 5′-end and or internal position is achieved by using appropriately protected ligand-phosphoramidite building block. An extended 15 min coupling of 0.1 M solution of phosphoramidite in anhydrous CH3CN in the presence of 5-(ethylthio)-1H-tetrazole activator to a solid-support-bound oligonucleotide. Oxidation of the internucleotide phosphite to the phosphate is carried out using standard iodine-water as reported (1) or by treatment with tert-butyl hydroperoxide/acetonitrile/water (10:87:3) with 10 min oxidation wait time conjugated oligonucleotide. Phosphorothioate is introduced by the oxidation of phosphite to phosphorothioate by using a sulfur transfer reagent such as DDTT (purchased from AM Chemicals), PADS and or Beaucage reagent. The cholesterol phosphoramidite is synthesized in house and used at a concentration of 0.1 M in dichloromethane. Coupling time for the cholesterol phosphoramidite is 16 minutes.

After completion of synthesis, the nucleic acid molecule is cleaved with simultaneous deprotection of base and phosphate groups. The product is then further treated to remove the tert-butyldimethylsilyl (TBDMS) groups at the 2′ position for deprotection.

### Purification and Analysis

The synthesied RNA molecules is purified and analyzed by high-performance liquid chromatography (HPLC). The synthetic products are purified by anion-exchange HPLC on a TSK gel column. The buffers are 20 mM sodium phosphate (pH 8.5) in 10% CH₃CN (buffer A) and 20 mM sodium phosphate (pH 8.5) in 10% CH₃CN, 1M NaBr (buffer B). Fractions containing full-length oligonucleotides are pooled, desalted, and lyophilized. Approximately 0.15 OD of desalted oligonucleotidess are diluted in water to 150 μL and then pipetted into special vials for CGE and LC/MS analysis. Compounds are then analyzed by LC-ESMS and CGE.

Assembly of sgRNA Molecules

The oligonucleotides of the target recognistion sequences are annealed with a 80 nucleotides chimeric sgRNA core sequence ((5′-AAAAGCACCGACTCGGTGCCACTTTTTCAAGTTGATAACGGACTAGCCTTATTTT AACTTGCTATTTCTAGCTCTAAAAC-3′). The annealed oligonucleotides are then filled in using Phusion polymerase (New England BioLabs) under the following conditions: 98° C. for 2 min; 50° C. for 10 min; 72° C. for 10 min.

The quality of the assembled oligonucloetides is checked on a 2.5% agarose gel. The assembled sgRNA template is then used to transcribe RNA by in vitro transcription.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

1. A conjugate for editing a polynucleotide sequence in a cell or an organism comprising the structure of the formula X—Y—Z, wherein X is a targeting moiety; Y is an optional linker; and Z is a guide RNA.

2. The conjugate of claim 1, wherein the active agent, Z, is a single guide RNA (sgRNA).

3. The conjugate of claim 2, wherein the sgRNA is about 10 nucleotides to about 250 nucleotides in length.

4. The conjugate of claim 3, wherein the sgRNA is about 20 nucleotides to about 100 nucleotides in length.

5. The conjugate of claim 3, wherein the sgRNA comprises a polynucleotide sequence that consists of three regions: a target recognition sequence, a trans-activating crRNA (tracr) sequence and a sequence that is complementary to the tracr sequence.

6. The conjugate of claim 5, wherein the target recognition sequence of the sgRNA comprises about 12 to about 25 nucleotides that are complementary to and hybridize to the 12-25 consecutive nucleotides of a selected target polynucleotide in the genome of said cell or organism.

7. The conjugate of claim 6, wherein the target recognition sequence of the sgRNA comprises 15-20 nucleotides that are complementary to and hybridize to the 15-20 consecutive nucleotides of the selected target polynucleotide in the genome of said cell or organism.

8. The conjugate of claim 5, wherein the tracr sequence of the sgRNA comprises a wild type tracrRNA sequence identified from a bacteria strain in which a CRISPR-Cas system is identified, wherein the tracr sequence hybridizes to the sequence complementary to the tracr sequence that is linked to the target recognition sequence of the sgRNA, and forms part of a CRISPR-Cas complex.

9. The conjugate of claim 8, wherein the tracr sequence comprises about 20 to about 100 nucleotides.

10. The conjugates of claim 8, wherein the complementarity between the tracr sequence and the sequence that is complementary to the tracr sequence is between at least about 70% and at least 100%.

11. The conjugate of claim 6, wherein the selected target polynucleotide in the genome locates immediately at the 5′ end of a postspacer adjacent motif (PAM), wherein the PAM sequence is not included in the target recognition sequence of the sgRNA molecule.

12. The conjugate of claim 11, wherein the PAM comprising the sequence selected from the group consisting of NGG, NNGRRT, NNNGATT, NNNAGAAW and NNAAAC, wherein N represents any one of A, T, G, C. and W represents A or T.

13. The conjugate of claim 5, wherein the sgRNA molecule further comprises one or more additional nucleotides at the 5′ end of the RNA molecule that is not complementary to the selected target polynucleotide in the genome.

14. The conjugate of claim 13, wherein the sgRNA comprises one, or two, or three additional nucleotides at the 5′ end of the RNA molecule.

15. The conjugate of claim 5, wherein the sgRNA molecule comprises one or more modified nucleotides.

16. The conjugate of claim 5, wherein the complementarity between the target recognition sequence of the sgRNA molecule and the selected target polynucleotide is from about 70% to about 100%.

17. The conjugate of claim 1, wherein the linker is a cleavable linker.

18. The conjugate of claim 17, wherein the linker is enzymatic-cleavable.

19. The conjugate of claim 17, wherein the linker is non-enzymatic cleavable.

20. The conjugate of claim 17, wherein the linker is selected from the group consisting of an alkyl chain, a peptide, a beta-glucuronide, a self-stabilizing group, a hydrophilic group and a disulfate group.

21. The conjugate of claim 1, wherein the targeting moiety and the active agent of the conjugate are directly connected.

22. A nanoparticle for editing a polynucleotide in a genome of a cell or an organism comprising
(i) at least one conjugate comprising the structure of the formula X—Y—Z, wherein X is a targeting moiety; Y is an optional linker; and Z is a guide RNA; and
(ii) at least one Cas protein.

23. The nanoparticle of claim 22, wherein the nanoparticle comprises a polymeric matrix.

24. The nanoparticle of claim 23, wherein the polymeric matrix comprises one or more polymers selected from the group consisting of hydrophobic polymers, hydrophilic polymers, and copolymers thereof.

25.-26. (canceled)

27. The nanoparticle of claim 23, wherein the polymeric matrix comprises one or more polymers selected from the group consisting of poly(lactic acid), poly(glycolic acid), poly(lactic-co-glycolic acid), poly(ethylene oxide), poly(ethylene glycol), poly(propylene glycol), and copolymers thereof.

28. The nanoparticle of claim 23, wherein the size of the nanoparticle is between 10 nm and 5000 nm.

29. (canceled)

30. The nanoparticle of claim 23, wherein the weight percentage of the conjugate is between 0.1% and 35%.

31. The nanoparticle of claim 22, wherein the Cas protein is selected from Cas9 or Cpf1.

32. A composition for editing a polynucleotide in a cell or an organism comprising the conjugate of claim 1 and a Cas protein.

33. The composition of claim 32, wherein the Cas protein is selected from Cas9 or Cpf1.

34. A method for editing a selected polynucleotide in a cell or in a subject, the method comprising
(i) introducing into the cell, or the subject, of at least one conjugate as defined in claim 1, and
(ii) at least one Cas protein,
wherein the CAS protein is introduced into the cell or the subject as a polypeptide, or its variants, a RNA molecule that encodes the Cas protein or its variants, a construct including a nucleic acid molecule that encodes the Cas protein or its variants, or an expression vector that is used to express the Cas protein or its variants.

35. The method of claim 34, wherein the Cas protein is a Type II CRISPR Cas9 endonuclease or a variant thereof.

36. The method of claim 34, wherein the Cas protein is Cpf1 or a variant thereof.

37. A method for editing a selected polynucleotide in a cell, or in a subject, the method comprising introducing into the cell, or the subject, of a nanoparticle of claim 22, or a composition of claim 31.

38. The method of claim 35, wherein the variants of the Cas9 endonuclease include Cas9 proteins isolated from other bacterial strains, a Cas9 nickase having one inactive nuclease domain, a nuclease-null dead Cas9 protein (dCas9), and a fusion protein comprising a dCas9 protein is fused with one or more heterogeneous effector domains.

39. The method of claim 34, wherein the selected polynucleotide in the cell or the subject locates immediately at the 5′end of a postspacer adjacent motif (PAM) that is specifically recognized by a Cas9 nuclease.

40. The method of claim 36, wherein said one or more heterogeneous effector domains fused with the dCas9 protein comprise domains having activities of transcriptional activation; transcription suppression, methylase activity, demethylase activity, histone modification activity, RNA cleavage activity and nucleic acid binding activity, and chromatin modification activity.

41. The method of claim 36, wherein said one or more heterogeneous effector domains fused with the dCas9 protein comprises epitope tags and reporter gene sequences.


SEQUENCE LISTING (1 sequence)

SEQ ID NO: 1 - DNA, 80 bp, Artificial Sequence
  Description of Artificial Sequence Synthetic polynucleotide
aaaagcaccgactcggtgccactttttcaagttgataacggactagccttattttaacttgctatttctagctctaaaac